(19) United States
(12) Patent Application Publication
(10) Pub. No.: US 2019/0293627 A1
(43) Pub. Date: Sep. 26, 2019

(54) APPARATUS AND METHOD FOR PERSONALIZING NUTRITION BASED ON BIOSENSOR DATA

(71) Applicant: Bender Tech, LLC, Raleigh, NC (US)

(72) Inventor: Brian Francis Bender, Los Angeles, CA (US)

(21) Appl. No.: 16/430,553

(22) Filed: Jun. 4, 2019

Related U.S. Application Data
(63) Continuation of application No. PCT/US17/64595, filed on Dec. 5, 2017.
(60) Provisional application No. 62/430,077, filed on Dec. 5, 2016.

(51) Int. Cl. G01N 33/493; G16H 20/60

(52) U.S. Cl. CPC G01N 33/493; G16H 40/63; G16H 20/60

(57) ABSTRACT

The presently disclosed subject matter is directed to a system and method of using electrochemical spectroscopy to detect and quantify concentrations of biomarkers of dietary intake and physiological health in urine samples. This presently disclosed subject matter can be used to quantify nutrition and disease biomarkers, detect dietary deficiencies, and/or report the levels back to the user. In some embodiments, the disclosed system and method can be expanded to multiplexed systems used for a wide variety of dietary nutrient biomarkers to direct diet and/or create custom therapies.

### CROSS REFERENCE TO RELATED APPLICATIONS

This application is a continuation of International Patent Application No. PCT/US17/64595, filed on Dec. 5, 2017, which claims priority to and the benefit of U.S. Provisional Patent Application No. 62/430,077, filed on Dec. 5, 2016, which is incorporated by reference herein in its entirety.

### TECHNICAL FIELD

The presently disclosed subject matter relates generally to an apparatus and method for personalizing nutrition based on biosensor data.

### BACKGROUND

As one in three global citizens suffer from malnutrition and dietary risks comprise the greatest disease risk in the U.S., the USDA, the NIH, and the WHO have all identified better data collection methods as a primary need to improve global nutritional health outcomes. (Institute, I. F. P. R. Global Nutrition Report 2016: From Promise to Impact: Ending Malnutrition by 2030. (2016); Murray, C. J. L. The State of US Health, 1990-2010 Burden of Diseases, Injuries, and Risk Factors. JAMA 310, 591-606 (2013)). In fact, the United Nations General Assembly agreed to a resolution proclaiming the UN Decade of Action on Nutrition from 2016 to 2025, aiming to intensify action to eradicate malnutrition worldwide and incorporating dietary risks as part of 12 of the 17 UN Sustainable Development Goals. Furthermore, it is becoming evident that nutritional needs are highly personalized, and detection of personal metabolic activity will be key to improving health outcomes.

Constituents of nutritional health can be categorized broadly into macronutrients and micronutrients. Macronutrients include higher-order nutritional categories such as carbohydrates, proteins, and fats. Micronutrients include the individual vitamins, minerals, and other peptides and proteins derived from dietary intake that are necessary for physiological health and well-being. Vitamins and minerals are largely consumed from an individual's diet, but deficiencies can occur when they are not regularly consumed at adequate levels or during periods of atypical physical stress. The American population largely consumes most micronutrients at sub-clinical levels according to the CDC's NHANES survey data.

Blood sampling is invasive and is therefore performed infrequently, if at all. Furthermore, it requires a clinician and can often be expensive. One ramification is the dearth of nutritional data among the population, particularly in the developing world. Analytical methods of biomarker quantification are also not ideal, requiring large, expensive equipment and trained technical operators. Thus, rapid, inexpensive, convenient, and non-invasive tools for quantifying nutritional biomarkers would beneficially help individuals and institutions better assess the nutritional health of the population and provide personalized dietary changes or supplements to address an individual's nutritional needs.

Correlations have been established for several urinary biomarkers associated with dietary intake, including (but not limited to) vitamin B1, vitamin B2, vitamin B3, vitamin B5, vitamin B6, vitamin B7, vitamin B9, vitamin B12 (via methylmalonic acid), vitamin C, vitamin A, vitamin K, and Vitamin E (via α-CEHC). (Yoshida, M., Fukuwatari, T., Sakai, J., Tsuji, T. & Shibata, K. Correlation between Mineral Intake and Urinary Excretion in Free-Living Japanese Young Women. 2012, 123-128 (2012); Fraser, G. E. et al. Biomarkers of Dietary Intake Are Correlated with Corresponding Measures from Repeated Dietary Recalls and Food-Frequency Questionnaires in the Adventist Health Study-2. J. Nutr. 146, 586-594 (2016); Shibata, K., Hirose, J. & Fukuwatari, T. Relationship Between Urinary Concentrations of Nine Water-soluble Vitamins and their Vitamin Intakes in Japanese Adult Males. Nutr. Metab. Insights 61 (2014); Sorkhi, H. & Aahmadi, M. H. Urinary calcium to creatinin ratio in children. Indian J. Pediatr. 72, 1055-1056 (2005); Rohner, F. et al. Biomarkers of Nutrition for Development—Iodine. J. Nutr. 144, 1322S-13425S (2014)). Further, vitamin D may have a correlation to some urinary biomarker yet to be discovered. For example, calcitroic acid is an end metabolic product in urine and may correlate to vitamin D intake. Additionally, several minerals have been shown to correlate well to dietary intake, including sodium, iodine, potassium, calcium, magnesium, phosphorus, selenium, and molybdenum. (Yoshida, M., Fukuwatari, T., Sakai, J., Tsuji, T. & Shibata, K. Correlation between Mineral Intake and Urinary Excretion in Free-Living Japanese Young Women. 2012, 123-128 (2012)). Hepcidin-25 may also correlate to iron intake. The cited vitamins and minerals (including others not listed) can be detected and quantified using the methods described herein. In addition, various biomarkers of nutrient metabolism for each of the listed nutrients (and others not listed) can be detected to further monitor nutritional health. For example, methylmalonic acid can be used for monitoring vitamin B12 stores and metabolism, 3-hydroxyisovaleric acid for biotin metabolism, 3-hydroxykynurenine (3-HK), and xanthurenic acid (XA) for functional B6 metabolism. (Ueland, P. M., Ulvik, A., Rios-Avila, L., Midttun, Ø. & Gregory, J. F. Direct and Functional Biomarkers of Vitamin B6 Status. Annu. Rev. Nutr. 35, 33-70 (2015)). Other biomarkers related to nutritional intake and health can be quantified, including (but not limited to) ketones and specific amino acids.

The apparatus and methods disclosed herein can be extended into biomarkers for general diet tracking, macronutrient detection, and other markers of nutrition. For example, an extensive comparison of serum and urinary biomarkers to macronutrient dietary intake patterns has been developed. (Playdon, M. C. et al. Comparing metabolite profiles of habitual diet in serum and urine 1-3. Am. J. Clin. Nutr. (2016); Yin, X. et al. Estimation of Chicken Intake by Adults Using Metabolomics-Derived Markers. J. Nutr. 147, 1850-1857 (2017)). The metabolite profile can be used to identify biomarkers of dietary patterns, such as red meat intake, chicken intake, added-sugar, leafy green vegetables, caffeine, alcohol intake, processed meats, fish intake, grain intake, dairy intake, and others. Such information can help track habitual diet, suggest dietary changes, and identify discrepancies between nutrient intake and biomarker excretion that may indicate underlying metabolic disturbances that derive from genetic, disease, or environmental factors. Additionally, urinary and/or fecal metabolites can act as tools for measuring gut microbiota composition, function, and dynamics for nutritional and metabolic health. (Meyer, T. W. & Hostetter, T. H. Uremic solutes from colon microbes. Kidney Int. 81, 949-954 (2012); Sonnenburg, J. L. & Bäckhed, F. Diet—microbiota interactions as moderators of human metabolism. Nature 535, 56-64 (2016); Kobyliak, N., Virchenko, O. & Falalyeyeva, T. Pathophysiological role of host microbiota in the development of obesity. Nutr. J. 1-12 (2016)).

Macronutrient (protein, carbohydrates, and fats) and total energy intake (calories) are also important constituents for ongoing monitoring and optimization to prevent many poor health outcomes like obesity, diabetes, cardiovascular disease, high blood pressure, several cancers, and other diseases. Urea is produced through the metabolism of amino acids. The concentration of urea in urine (along with other nitrogenous compounds like uric acid and ammonia) correlate to recent protein intake. Urinary fructose and/or sucrose correlate to recent dietary sugar intake, 3-(3,5-dihydroxyphenyl)-propanoic acid correlates to grain fiber intake, and acetylcarnitine correlates to red meat intake. Polyamines are a class of compounds that are present in all organic matter and are associated with cellular metabolism. Research has shown an association of urinary polyamines with metabolism and energy intake, as well as certain dietary intake patterns. For example, the sum of putrescine, spermidine, and spermine has been correlated to total dietary fat intake, and N8-acetylspermidine has been correlated with total energy intake.

The large number of competitors in the diet and nutrition space indicates a robust market demand for nutrition testing, tracking, and optimization services. According to a survey by the American Dietetic Association, roughly 46.0% of the population are actively searching for information about nutrition and healthy eating. (IBIS World. Increases in obesity and diabetes will drive demand for industry services in the US: Nutritionists & Dietitians. (2014)). However, a solution for accurate, physiological data in an easy-to-use device is still lacking. Food journal apps and surveys are notoriously inaccurate and have poor long-term adherence. Although some popular food journal apps have garnered over 160 million registered users, studies have shown that up to 97% of them stop using it after only one week. (Helander, E., Kaipainen, K., Korhonen, I. & Wansink, B. Facors Related to Sustained e of a Free Mobile App for Dietary Self-Monitoring With Photography and Peer Feedback: Retrospective Cohort Study. J. Med. Int. Res. 16, e109 (2014)). The reasons for quitting are often associated with the tedium of data collection. For example, users do not like inputting food data after every meal, sometimes users forget meals and thus renders the day's data less meaningful, and inputting home-cooked meals can be difficult. Further, while blood testing services are accurate, they are invasive, require a trip to a clinic, and are thus rarely used by the broader consumer market. In addition, surveys are typically error prone, and one-on-one nutrition counseling is viewed as a luxury that does not provide physiologically relevant data. Saliva-based DNA testing is an emerging and competitive market for nutrition, but at best can only suggest dietary regimes (i.e., they cannot track recent dietary intake or micronutrient deficiencies). It would therefore be beneficial to provide an improved apparatus and method for nutrition testing, tracking, and optimization.

### SUMMARY

In some embodiments, the presently disclosed subject matter is directed to a biosensor apparatus comprising at least one working electrode comprising an outside surface. In some embodiments, at least one affinity probe is present on the outside surface of the working electrode. In some embodiments, the affinity probe can be a g molecule and/or a chemical. Alternatively, the outside surface of the working electrode can be free from bound affinity probe. The affinity probe targets one or more urinary biomarkers, such as a nutritional biomarker and/or a biomarker associated with a disease or disorder (e.g., kidney disease). The apparatus further comprises an electrochemical spectroscopy analyzer.

In some embodiments, the presently disclosed subject matter is directed to a method of obtaining data on the nutritional health of a subject. Particularly, the method comprises contacting a solution comprising a subject's urine with a working electrode of a biosensor apparatus comprising at least one working electrode with an outside surface that comprises at least one affinity probe or an outside surface free from affinity probe. The biosensor apparatus further comprises an electrochemical spectroscopy analyzer. The method comprises performing electrochemical spectroscopy to generate data for the working electrode, using the data to quantify an amount of at least one nutritional biomarker in the subject's urine, and correlating the amount of the at least one nutritional biomarker to obtain nutritional health data.

In some embodiments, the presently disclosed subject matter is directed to a method of obtaining health data on the presence of a disease or disorder of a subject. Particularly, the method comprises contacting a solution comprising a subject's urine with a working electrode of a biosensor apparatus. The biosensor apparatus comprises at least one working electrode that includes at least one affinity probe present on the outside surface of the working electrode, or the outside surface of the working electrode is free from affinity probe. The biosensor apparatus further comprises an electrochemical spectroscopy analyzer. The method comprises performing electrochemical spectroscopy to generate data for the working electrode, using the data to quantify the concentration of at least one biomarker associated with the presence of the disease or disorder in the subject's urine. and correlating the concentration of the at least one biomarker to obtain health data related to the disease or disorder.

In some embodiments, the presently disclosed subject matter is directed to a method of diagnosing a condition or ailment in a subject. Particularly, the method comprises contacting a solution comprising a subject's urine with a working electrode of a biosensor apparatus wherein the biosensor apparatus comprises at least one affinity probe is present on the outside surface of the working electrode or the outside surface of the working electrode is free from affinity probe. The biosensor apparatus further comprises an electrochemical spectroscopy analyzer. The method comprises performing electrochemical spectroscopy to generate data for the working electrode; using the data to quantify the amount of at least one nutritional biomarker in the subject's urine; correlating the amount of the nutritional biomarker to obtain nutritional health data; and using the nutritional health data to diagnose a condition or ailment. In some embodiments, the condition or ailment can include non-disease states, such as a state of dietary intake not optimal for the user based on their own decision, a recommended daily allowance, a nutritionist, etc.

In some embodiments, the electrodes comprise a reference electrode, a counter electrode, and more than one working electrode. In some embodiments, the electrodes comprise a working electrode and a second electrode serving as both the reference electrode and the counter electrode. In some embodiments, at least one electrode is used to specifically target creatinine.

In some embodiments, the affinity probe is an antibody, aptamer, affimer, hapten, enzyme, chemical, or combinations thereof.

In some embodiments, the affinity probe can be a targeting chemical selected from copper oxide particles, nickel oxide particles, ferricyanide, ionophores, polymeric matrices, lanthanum manganite, nanofibers, or combinations thereof.

In some embodiments, the affinity probe is bound to the working electrode using physioabsorption, covalent bonding, ionic bonding, physical entrapment, cross-linking, encapsulation, disulfide linking, chelation, metal binding, hydrophobic binding, or combinations thereof.

In some embodiments, the apparatus further comprises additional working electrodes with an affinity probe bound thereto, wherein each working electrode binds a separate and distinct urinary biomarker, or produces a reaction product that is detected.

In some embodiments, the biomarker is vitamin B1 (thiamin), vitamin B2 (riboflavin), vitamin B3 (niacin), vitamin B4, vitamin B6, vitamin B7 (biotin), vitamin B9 (folate), vitamin C, methylmalonic acid, vitamin A, vitamin E, vitamin D, vitamin K, sodium, potassium, calcium, magnesium, phosphorous, selenium, molybdenum, chromium, zinc, copper, iodine, iron, manganese, 1-methylhistidine, creatinine, catabolites, metabolites, urea, N8-acetylspermidine, putrescine, spermidine, spermine, albumin, or combinations or vitamers thereof.

In some embodiments, the electrodes are screen printed, ink-jet printed, sputtered, or evaporated on a substrate.

In some embodiments, the substrate is constructed from paper, ceramics material, polymeric material, or combinations thereof.

In some embodiments, the apparatus further comprises a housing that encloses the electrodes, wherein the housing comprises a window (e.g., a screen) to allow exposure of a desired area of the electrodes, and wherein the housing is configured to move along the length of the electrode.

In some embodiments, the apparatus further comprises a rotation element around which the substrate is incrementally wrapped during operation.

In some embodiments, the amount of the at least one nutritional biomarker comprises a correlation to intake of a dietary micronutrient, dietary macronutrient, dietary food group, total caloric intake, other dietary constituent, or combinations thereof.

In some embodiments, the dietary micronutrient can be selected from one or more vitamins, mineral, fish and plant oils, amino acids, enzymes, phytochemicals, herb and fruit extracts, etc. In some embodiments, the dietary macronutrient can be selected from one or more of fat, protein, carbohydrate, etc. In some embodiments, the dietary food group can be selected from vegetables, legumes/beans, fruit, grain foods, meat (poultry, fish, etc.), dairy (milk, yogurt, cheese), and/or nuts/seeds. Total caloric intake refers to the total amount of calories consumed by an individual in a day.

In some embodiments, the disclosed method(s) further comprise comparing the subject's nutritional health data to recommended daily allowances, intake levels established by the subject, intake levels established by a dietitian, physician, or nutritionist, intake levels established algorithmically, the subject's previously measured intake data, and/or intake levels of segmented populations for a particular nutritional biomarker.

In some embodiments, the health data is selected from impedance data, amperometric data, voltammetric data, potentiometric data, conductometric data, or combinations thereof.

In some embodiments, the health data is used to monitor the disease or disorder and/or is used to diagnose the disease or disorder by correlating to known health data for the disease or disorder.

In some embodiments, the health data can be collected by measuring: impedance, using correlations between impedance and biomarker concentration to obtain biomarker concentration, and comparing the biomarker concentration to correlations between biomarker concentration and a disease or disorder; voltammetric data, using current response to changing voltage; amperometric data, using current response to a fixed voltage; potentiometric data, using voltage response in relation to another electrode; conductometric data, using electrical conductivity or current flow response due to a change ionic species separation; or combinations thereof.

In some embodiments, the nutritional biomarker comprises a correlation to a clinical pathology and is used to diagnose a pathology, monitor a pathology or both. The term “pathology” as used herein refers to any deviation from a healthy or normal condition, such as a disease, disorder, syndrome, or any abnormal medical condition.

In some embodiments, the nutritional health data can be used to provide customary food, dietary, or both food and dietary suggestions, develop a custom medication, or both.

### BRIEF DESCRIPTION OF THE DRAWINGS

The previous summary and the following detailed descriptions are to be read in view of the drawings, which illustrate some (but not all) embodiments of the presently disclosed subject matter.

FIG. 1a is a perspective view of a sensing device in accordance with some embodiments of the presently disclosed subject matter.

FIG. 1b is an exploded view of the device of FIG. 1a.

FIG. 2a is a top plan view of a sensing strip in accordance with some embodiments of the presently disclosed subject matter.

FIG. 2b is a top plan view of a sensing strip in accordance with some embodiments of the presently disclosed subject matter.

FIGS. 2c and 2d are perspective views of a sensing strip and a coordinating connector in accordance with some embodiments of the presently disclosed subject matter.

FIGS. 3a-3c are perspective views of a sensor and housing in accordance with some embodiments of the presently disclosed subject matter.

FIGS. 4a and 4b are perspective views of an abrasive element that can be used in accordance with some embodiments of the presently disclosed subject matter.

FIGS. 5a and 5b are perspective views of representative electrodes in accordance with some embodiments of the presently disclosed subject matter.

FIG. 5c is a perspective view illustrating a mechanism of moving the substrate and/or housing to expose new regions of the electrodes.

FIG. 6a is a perspective view a sensor strip movement mechanism comprising rollers controlled by a micro-stepper and motor and gears.

FIG. 6b is a perspective view illustrating a roller mechanism that covers electrode regions not in use.

FIGS. 7a-7e illustrate various embodiments wherein the substrate comprising the electrodes is moved incrementally using a gear, rotor, or other rotation element attached to the distal end of the substrate.

FIG. 8a is a perspective view of a device comprising flaps to prevent water backflow.

FIG. 8b is a perspective close up view of the device of FIG. 8a.

FIG. 9a is a perspective view of and device that houses disposable strips.

FIGS. 9b-9d are perspective views of toilets housing the device of FIG. 9a.

FIGS. 10a and 10b are perspective view of a housing that includes the electrodes.

FIG. 11a is a Bode plot of impedance (Z′) versus frequency (Hz) for water and water+vitamin B7.

FIG. 11b is a Nyquist plot of imaginary impedance (−Z″) versus real impedance (Z′) for water and water+vitamin B7.

FIG. 12a is a Bode plot of frequency (Hz) versus magnitude of impedance (Z) for biotin in diluted urine samples.

FIG. 12b is a Nyquist plot of imaginary impedance (−Z″) versus real impedance (Z′) for biotin in diluted urine samples.

FIG. 13 is a plot of impedance versus the concentration of biotin in diluted urine. FIG. 14a is a plot of cyclic voltammetry taken before and after exposure of 1% copper I oxide embedded working electrodes to buffer and urine samples with and without creatinine.

FIG. 14b is a plot illustrating the linear correlation between creatinine concentration and peak height.

FIG. 15a is a voltammogram taken before and after exposure of 5% nickel oxide embedded working electrodes to buffer and urine samples with and without urea.

FIG. 15b is a plot illustrating the linear correlation between urea concentration and current at various voltages.

FIG. 15c is a plot of linear voltammetric measurement carried out from −0.5V to 1.5 using a scan rate of 25 mV/s.

FIG. 16a is a voltammogram taken before and after exposure of working electrodes with 1 mM ferricyanide deposited and dried on their surface to PBS buffer with and without albumin.

FIG. 16b is a linear sweep voltammogram illustrating the effect of BSA on 1 mM ferricyanide in PBS.

FIG. 16c is a plot of the linear correlation between albumin concentration and current at various concentrations.

FIG. 17a is a cyclic voltammetry plot of differing BSA concentrations present in 1 mM ferricyanide solution containing L-cysteine.

FIG. 17b is a cyclic voltammetry plot of differing BSA concentrations present in solution containing L-cysteine (no ferricyanide).

FIG. 18a is a cyclic voltammetry plot of a working electrode modified with 3% Hematein and increasing albumin concentration.

FIG. 18b is a cyclic voltammetry plot of a working electrode modified with 2% lauryl gallate and increasing albumin concentration.

FIG. 19a is a voltammogram plot taken before and after exposure of LaMnO₃ nanofiber coated working electrodes to buffer and urine samples with and without fructose.

FIG. 19b is a plot of amperometric detection using fructose dehydrogenase+3 mM ferricyanide coated working electrodes.

FIG. 19c is a plot of the correlation between fructose concentration and steady state current.

FIG. 20a is plot of the voltammograms taken before and after exposure of p-AMTa modified electrodes to buffer and diluted urine spiked with vitamins B2, B9, and vitamin C.

FIG. 20b is a plot of the correlation between ascorbic acid concentration and peak current.

FIG. 21 is a voltammogram taken before and after exposure of p-AMTa modified electrodes to buffer and diluted urine spiked with vitamins B2, B9, and vitamin C.

FIGS. 22a and 22b are voltammograms taken before and after exposure of PEDOT/ZrO2NP modified electrodes to buffer and diluted urine spiked with vitamins B2, B9, and vitamin C, before and after riboflavin addition.

FIG. 22c is a plot of the linear correlation between riboflavin concentration and peak current.

### DETAILED DESCRIPTION

The presently disclosed subject matter is presented with sufficient details to provide an understanding of one or more particular embodiments of broader inventive subject matters. The descriptions expound upon and exemplify particular features of those particular embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “an electrode” can include a plurality of such electrodes, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

The presently disclosed subject matter is directed to electrochemical biosensors and methods of using the disclosed biosensors to measure a wide array of biomarkers for a variety of clinical indications. The disclosed system and method comprise the use of biosensors to detect urinary biomarkers that can be correlated to a subject's nutritional health. Specifically, quantification can be used to diagnose a variety of conditions and ailments (such as metabolic disorders) and/or curate specific needs for the subject, such as the provision of food and dietary suggestions and/or custom supplement development.

It should be appreciated that the presently disclosed subject matter is not limited and the biosensors can include magnetoelastic biosensors, piezoelectric biosensors, chemiluminescence-based biosensors, fluorescence-based biosensors, surface-plasmon resonance-based biosensors, optical biosensors, microbial biosensors, Raman FTIR biosensors, fiber optic biosensors, field effect transistor-based biosensors, calorimetric biosensors, or surface acoustic wave biosensors. Further, a wide variety of biological fluids can be tested for nutritional biomarkers and/or disease-based biomarkers as set forth herein. For example, tears, sweat, blood, breath, saliva, fecal matter, blood plasma, and/or tissue biopsies can be tested.

To measure micronutrient levels, researchers and clinicians quantify biomarkers for each micronutrient in different conditions and using different methods. For example, urinary methylmalonic acid (MMA) concentration has been used to assess vitamin B12 stores, and urinary calcium is known to correlate to dietary intake. In addition, vitamin D deficiency is often assessed through blood serum quantification of 25-OHD using HPLC or mass spectrometry. Further, calcitroic acid is a metabolite of vitamin D and can provide a urinary biomarker for dietary intake levels. Hepcidin-25 has also been shown to have potential as a urinary biomarker of iron.

Advantageously, the presently disclosed apparatus and method can be used for general diet tracking, macronutrient detection, and/or other markers of nutrition. Alternatively or in addition, the disclosed apparatus can be used to track, detect, and/or otherwise mark one or more particular diseases or disorders (e.g., kidney disease). The metabolite profile can be used to identify biomarkers of dietary patterns, such as red meat intake, added-sugar, leafy green vegetables, and others. The information can help track a subject's habitual diet, suggest dietary changes, and/or identify discrepancies between nutrient intake and biomarker excretion. Such information can indicate underlying metabolic disturbances that derive from genetics, disease, and/or environmental factors. For example, 3-hydroisovaleric acid (3-HIA) can be used to detect biotin metabolism. Accordingly, tracking both biotin and its derivatives measured against 3-HIA can help understand metabolic health for biotin. Similarly, catabolites (such as p-aminobenzoylgultamate) can help diagnose the metabolic activity of folate.

Thus, the disclosed device can be used to detect and measure a variety of urinary biomarkers for macronutrient detection and various important nutritional food categories, such as sugar and red meat. In addition to total caloric intake, protein intake, and fat intake, total carbohydrate intake can be determined by subtracting protein and fat intake from total caloric intake. Other food categories, vitamins, and minerals can be determined using this framework as well. Creatinine can be used to normalize each biosensor measurement. Specifically, the final measurement used to determine dietary intake is the ratio of the desired analyte to the concentration of creatinine measured, as shown below:

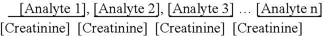

Creatinine is a known metabolic breakdown product of muscle tissue and is processed and excreted by the kidneys. It is a measure of renal function and is produced at a fairly constant rate. Creatinine is used as a normalization factor for several clinically validated urine tests. Normalization is necessary to compare different testing conditions and concentrations. For example, different toilets contain different baseline water volumes, and urine output varies per void. Therefore, the final concentration will vary by an unknown amount, rendering direct quantification rather meaningless in spot samples without normalization.

Similarly, N8-acetylspermidine can be used to measure caloric intake. N8-acetylspermidine has been found to possess a statistically significant positive correlation to total energy (kcal) intake (r=0.46, P=0.01). (Vargas, A. J. et al. Dietary Polyamine Intake and Polyamines Measured in Urine Dietary Polyamine Intake and Polyamines Measured in Urine. Nutr. Cancer 66, 1144-1153 (2017)). As energy intake requirements vary based on gender, age, daily activity, and basal metabolic rate, caloric requirement ranges typically vary between 200 and 400 calories a day and therefore provide a useful range for caloric intake measurements.

Urea can be used to measure dietary protein intake. Urea is a clinically validated biomarker for protein intake with decades of use and is correlated to protein intake via the equation below (although other equations exist and may be used), where p is daily protein intake in grams and w is body weight in kg. (Maroni, B. J., Steinman, T. I. & Mitch, W. E. A method for estimating nitrogen intake of patients with chronic renal failure. Kidney Int. 27, 58-65 (1985)). A confidence interval of 95% provides a roughly 0.2% variance in measured caloric intake derived from protein.

P=([Urea]+(w*0.031))*6.25

In some embodiments, nickel oxide can be incorporated into a carbon ink electrode (as set forth in more detail herein below) at 0.5-10 wt % (e.g., 5%) for urea detection. Sodium hydroxide or potassium hydroxide can be added to the working sensor at 1-10M (e.g., 5M). Polymers can be used to immobilize the hydroxide-containing chemicals, such as poly(ethylene oxide), polyvinyl acetate, polyvinylpyrrolidone, or any other polymer or combination thereof. A channelized biosensor design can be used to physically direct fluid flow via capillary forces for localized conditions suitable for electrochemical biomarker detection. Similarly, a mesh, membrane, or other absorbable material can be used to absorb fluid, direct fluid, and/or immobilize materials required for testing on an electrode or near an electrode. For example, some chemicals or materials can be used to adjust the local pH near the electrode to optimize the reaction. In addition, other materials may be used to adjust the reaction chemistry to better match that of biological fluids. For example, combining ascorbic acid with nickel oxide can provide a mechanism for enabling urea detection and quantification at a pH of about 7.

Continuing, the combined sum of the concentrations of urinary putrescine, spermidine, and spermine can be used to quantify total dietary fat intake. The combined concentration of these analytes possessed a statistically significant positive correlation to total fat intake (r_{adj}=0.41, P=0.02). (Vargas, A. J. et al. Dietary Polyamine Intake and Polyamines Measured in Urine Dietary Polyamine Intake and Polyamines Measured in Urine. Nutr. Cancer 66, 1144-1153 (2017)).

Sucrose, fructose, and/or sucrose plus fructose can be used to determine total dietary sugar intake. The urinary concentration of sucrose, fructose, and the combined sum of urinary sucrose+fructose all possess a statistically significant positive correlation to total dietary sugar intake (r=0.802, P<0.001). Fructose dehydrogenase (FDH), Invertase (Sucrase), Ferricyanide, L-cystein modified silver, arsezano I, and LaMnO₃ nanofibers (among others) can be used to detect fructose and sucrose.

Dihydroxyphenylpropionic acid (DHPPA) can be used to determine cereal fiber intake, including fiber from rye, wheat, and barley. Urinary DHPPA possesses a statistically significant positive correlation to cereal fiber intake (r=0.402, P=0.002). (Aubertin-leheudre, M., Koskela, A., Marjamaa, A. & Adlercreutz, H. Short Communication Plasma Alkylresorcinols and Urinary Alkylresorcinol Metabolites as Biomarkers of Cereal Fiber Intake in Finnish Women. Cancer Epidemiol Biomarkers Prev 17, 2244-2249 (2008); Aubertin-leheudre, M., Koskela, A., Samaletdin, A. & Adlercreutz, H. Responsiveness of Urinary and Plasma Alkylresorcinol Metabolites to Rye Intake in Finnish Women. Cancers (Basel). 2, 513-522 (2010)).

Acetylcarnitine can be used to determine red meat intake. Acetylcarnitine possesses a statistically significant positive correlation to red meat intake (r=0.32, P³=3.05×10⁻⁷). (Playdon, M. C. et al. Comparing metabolite profiles of habitual diet in serum and urine 1-3. Am. J. Clin. Nutr. (2016)).

In some embodiments, a suitable biomarker for dietary micronutrients can be selected from vitamin B1, vitamin B2, vitamin B3, vitamin B5, vitamin B6, vitamin B7, vitamin B9, vitamin C, calcitroic acid, methylmalonic acid, vitamin A, vitamin E, vitamin D, vitamin K, pantothenate, alpha-Tocopherol, pyridoxate, alpha-CEHC glucuronide, gamma-tocopherol, alpha-CEHC sulfate, sodium, potassium, calcium, magnesium, phosphorous, selenium, molybdenum, chromium, zinc, copper, iodine, iron, hepcidin-25, chloride, vanadium, boron, manganese, or catabolites, metabolites, equivalents, vitamers, ions, complexes, or combinations thereof.

In some embodiments, a suitable biomarker for total caloric intake can be selected from N8-acetylspermidine, N1-acetylspermidine, putrescine, spermidine, spermine, catabolites, metabolites, equivalents, or combinations thereof.

In some embodiments, a suitable biomarker for dietary macronutrients can be selected from N8-acetylspermidine, N1-acetylspermidine, putrescine, spermidine, spermine, urea, uric acid, ammonia, nitrogen, creatinine, creatine, or catabolites, metabolites, equivalents, or combinations thereof.

In some embodiments, a suitable biomarker for dietary food groups or other dietary constituents can be selected from coenzyme Q10, lipoic acid, L-carnitine, N-acetylcysteine, glutathione, creatine, 1-methylhistidine, 3-methylhistidine, creatinine, urea, N8-acetylspermidine, N1-acetylspermidine, N1,N12-diacetylspermidine, putrescine, spermidine, spermine, genistein, daidzein, glycitein, enterolactone, hydroxytyrosol, anthocyanins, hesperidin, naringenin, epicatechin,epigallocatechin, quercetin, dihydrodaidzein, equol, O-desmethylangolensin, isoflavones, isothiocyanate, 3,5-dihydroxybenzoic acid, 3-(3,5-dihydroxyphenyl)-1-propanoic acid, para-aminobenzoylglutamate, para-acetamidobenzoylglutamate, stachydrine, ethanol, uric acid, ammonia, dopamine, glutamine, isoprostanes, guanidoacetate, scyllo-inositol, deoxycarnitine, chiro-inositol, N-methylproline, betonicine, tryptophan betaine, mannitol, pimelate (heptanedioate), cytosine, suberate, homovanillate sulfate, kynurenin, CMPF, DHA (22:6n-3), 4-vinylphenolsulfae, acetylcarnitine, xylitol, 3-dehydrocarnitine, ethylglucuronide, lysine, ciliatine (2-aminoethylphosphonate), 2-hydroxybutyrate, N-acetyltyrosine, N-acetylglutamine, pantothenate, alpha-Tocopherol, pyridoxate, alpha-CEHC glucuronide, gamma-tocopherol, alpha-CEHC sulfate, quinate, paraxanthine, theophylline, 1-methylxanthine, trigonelline (N′-methylnicotinate), 1-methylurate, 1,3-dimethylurate, 5-acetylamino-6-formylamino-3-methyluracil, 1,7-dimethylurate, nicotinate, catechol sulfate, N-(2-furoyl) glycine, hippurate, 5-acetylamino-6-amino-3-methyluracil, 1,3,7-trimethylurate, pseudouridine, 1,7-dimethylurate, 3-methoxytyrosine, methylglutaroylcarnitine (3-methylglutarylcarnitine), glycerol 3-phosphate, ethyl glucuronide, 2,3-dihydroxyisovalerate, 2-isopropylmalate, nicotine, 2-hydroxy-N-(2-hydroxyphenyl)acetamide, N-(2-hydroxyphenyl)acetamide, enterolactone glucuronide, feruloyglycine sulfate, caffeic acid sulfate, feruloyglycine, ferulic acid sulfate, dihydroferulic acid sulfate, 2-aminophenol sulfate, 2-hydroxy-N-(2-hydroxyphenyl)acetamide sulfate, N-(2-hydroxyphenyl)acetamide sulfate, fructose, sucrose, creatinine, tyrosol, hydroxytyrosol, nitrogen, taurine, acetone, catabolites, metabolites, equivalents, or combinations thereof.

In some embodiments, the biomarker for detection, diagnosis, and monitoring of disease or disorder can be selected from albumin, apolipoprotein A-I, Alpha-1-microglobulin/bikunin precursor, heparan sulfate proteoglycans, malonate, N-methylnicotinamide, m-hydroxyphenylacetate, Hippuric acid, quinolinic acid, tyrosine, methylmalonic acid, putrescine, spermine, spermidine, N8-acetylspermidine, N1-acetylspermidine, azelaic acid, β-alanine, α-hydroxybutyrate, pseudouridine, 2,4-dihydroxypyrimidine, 2,4-dihydroxypyrimidine, choline, N-methylnicotinamide, oxalacetate, acetone, N-methylnicotinamide, N-methyl-2-pyridone-5-carboxamide, N-methyl nicotinic acid, taurine, N-methyl nicotinamide,

glutamate, fructose, 1,2,3-butanetriol, propylene glycol, phosphoric acid, sebacic acid, uric acid, pregnanediol, valine, glycine, glucose, creatinine, 3-(3-hydroxyphenyl)-3-hydroxypropanoic acid, ribose, transferrin, hemopexin, transthyretin, α2-HS-glycoprotein, Gc-globulin, α1-antitrypsin, kallikrein-binding protein, citrate, dimethylamine, glycine, hippurate, 3-hydroxykynurenine, homogentisate, allantoin, 5-hydroxyindoleacetic acid, cortisol, 11-deoxycortisol, 21-deoxycortisol, histidine, urocanic acid, imidazoleacetic acid, hydroxyphenylacetic acid, tyrosine, 1-methylnicotinamide, 2-oxoglutarate, citrate, urea, dimethylamine, trigonelline, trimethylamine, methionine, 5-hydroxyindoleacetic acid, desaminotyrosine, taurine, hydroxylauroylcarnitine, phenylacetic acid, histidine, dihydrocortisol, acetylserotonin, placenta-derived pregnancy-associated immunoregulatory proteins, choriogonadotropin subunit beta variant 1, fragments of collagen I, α-1(I) and α-1(III), fragments of uromodulin, prostate specific antigen 3, TMPRSS2:erg fusion, glutathione S-transferase P, glutamate carboxypeptidase 2, nuclear matrix protein 22 (NMP22), bladder tumor antigen, N1,N12 diacetyl spermine, N1,N8 diacetylspermidine, cytidine, 1-methyladenosine, adenosine, prostaglandin metabolite, aquaporin-1, adipophilin, osteonectin, profiling-1, polyubiquitin-C, Cyfra 21-1, leucine, leucine-rich repeat-containing protein 36, microtubule-associated serine/threonine-protein kinase 4, dynein heavy chain, axonemal, hemoglobin alpha-1, cytosol aminopeptidase, filaggrin, multimerin-2, agrin, neuronal growth regulator 1, fibrinogen alpha chain, extracellular matrix protein 1, catabolites, metabolites, or combinations thereof.

As set forth above, one or more affinity probes can be present (e.g., bound or otherwise attached) to the surface of the working electrode. Suitable affinity probes can include (but are not limited to) antibodies, aptamers, affimers, haptens, enzymes, chemicals and/or other natural or synthetic molecules that enable specific binding to a desired antigen.

The antibody, aptamer, or other targeting agent can be bound to an electrode surface in any manner known and used in the art to enable a chemical reaction with an analyte and produce a detectable electrochemical response. For example, in some embodiments, biotinylation, physical absorption, covalent binding, ionic binding, hydrophobic interactions, cross-linking, or entrapment can be used. Such methods are believed to be well-known to those of ordinary skill in the art.

The term “antibody” as used herein refers to a poplypeptide comprising a framework region from an immunoglobulin gene or fragments thereof that specifically binds and recognizes an antigen. In some embodiments, suitable antibodies that can be bound to the working electrode can include (but are not limited to) antibodies for hepcidin-25, urea, fructose, sucrose, creatinine, vitamin A, vitamin B1, vitamin B2, vitamin B3, vitamin B5, vitamin B6, vitamin B7, vitamin B9, methylmalonic acid, vitamin C, vitamin D, calcitroic acid, vitamin E, vitamin K, albumin, any of the nutritional or disease biomarkers listed above, any of the proteins listed above, and the like. The term “aptamer” as used herein refers to a single-stranded oligonucleotide (single-stranded DNA or RNA molecule) that can bind specifically to a target with high affinity. Particularly, the aptamers can be used as molecules targeting various organic and inorganic materials, including toxins. In some embodiments, suitable aptamers that can be bound to the working electrode can include (but are not limited to) aptmers that specifically target N8-acetylspermidine, putrescine, spermidine, spermine, any of the nutritional or disease biomarkers listed above or combinations thereof, any of the proteins listed above, and the like. The term “affimer” as used herein refers to small, highly stable proteins that bind to a target molecule with similar specificity and affinity to that of antibodies. In some embodiments, suitable affimers that can be bound to the working electrode can include (but are not limited to) affimers for any of the above listed biomarkers, for any of the above listed proteins, and the like. The term “hapten” as used herein refers to a partial antigen or non-protein binding member that is capable of binding to an antibody, but that is not capable of eliciting antibody formation unless coupled to a carrier protein. Suitable examples of haptens can include (but are not limited to) biotin, anti-biotin, avidin, and the like. The term “enzyme” as used herein refers to a protein that catalyzes at least one biochemical reaction. In some embodiments, suitable enzymes that can be bound to the working electrode can include (but are not limited to) N8-acetylspermidine oxidase, spermine oxidase, putrescine oxidase, invertase, glucose oxidase, fructose 5-dehydrogenase, urease, creatinine deiminase, creatinine kinase, creatinase, peroxidase, ascorbate oxidase, thiamine oxidase, diamine oxidase, cholesterol oxidase, uricase, microperoxidase, tyrosinase, monoamine oxidase, thiamine diphosphokinase, retinal oxidase, xanthine oxidase, NADPH oxidase, malic dehydrogenase, fumarase, succinic dehydrogenase, succinyl kinase, alpha-ketoglutaric dehydrogenase, isocitric dehydrogenase, aconitase, citric synthetase, ATP synthase, and/or other enzymes involved in the metabolism of dietary nutrients. The term “antigen” as used herein refers to the molecule recognized by a binding polypeptide (e.g., an antibody, aptamer, affimer, hapten, and/or enzyme).

Aptamers have favorable long-term stability compared to many biologically produced affinity probes, including antibodies and enzymes. In addition, aptamers have high specificity, are small, can be easily conjugated with redox probes and terminal linkers, and can be easily mass-produced once the aptamer is designed and isolated. Further, aptamers can also be designed to target multiple biomarkers at once.

Enzymes can also be used for selective binding. Enzymes are produced biologically or synthetically to specifically catalyze certain biochemical reactions. Like antibodies or aptamers, they generally have good specificity for certain molecules.

It should be understood that the antibodies, aptamers, affimers, and/or haptens disclosed herein include those that specifically interact with the cited biomarkers of interest. Such antibodies, aptamers, affimers, and/or haptens would be readily determined by those of ordinary skill in the art.

As set forth above, the affinity probe can include one or more chemicals. Suitable chemicals can be selected from 3-dimensional, 2-dimensional, 1-dimensional, tubule, or particle-based morphologies of platinum, palladium, gold, silver, mercury, iron, silver-chloride, boron-doped diamond, copper, bismuth, titanium, antimony, chromium, tin, nickel, aluminum, molybdenum, lead, tantalum, tungsten, steel, ruthenium oxide, indium tin oxide, bismuth oxide, co-Phthalocyanine, Meldola's blue, Prussian blue, biotin, streptavidin, avidin, extravidin, core quantum dots CdSe, core-shell quantum dots ZnS/CdSe, mesoporous carbon, carbon, graphite, graphene, graphene oxide, reduced graphene oxide, single-walled carbon nanotubes, multi-walled carbon nanotubes, double-walled carbon nanotubes, carbon nanofibers, copper oxide, nickel oxide, zinc oxide, manganese oxide, polyacetylene, polypyrrole, polyaniline, polythiopene, poly(3,4-ethylenedioxythiophene)-poly(styrenesulfonate, nafion, chitosan, poly(methyl methacrylate), poly(styrenesulfonic acid), ferricyanide, ferrocyanide, ferrocene derivatives, quinones, elemental ionophores, polyaniline, lanthanum manganese oxide, or oxidized states, ionic states, doped states, carboxyl-functionalized, derivatives, combinations of materials or morphologies thereof.

In some embodiments, additional chemicals and/or other materials can be used for specific sensing in lieu of or in addition to affinity probes. For example, silver nanoparticles, carbon nanotubes, copper (I) nanoparticles, copper (II) nanoparticles, gold nanoparticles, polyaniline, polyaniline nanoparticles, and/or any organic or inorganic material that reacts with the biomarkers of interest and/or byproducts thereof can be used. The term “nanotubes” as used herein refers to an elongated, hollow structure (such as an unfilled cylindrical shape) with a cross-section and/or diameter of less than about 1000 nm. The term “nanoparticle” as used herein refers to a particle having a largest dimension of less than about 500 nanometers. Labels and/or mediators can optionally be included in the design to promote more efficient electron transfer and signal amplification. Suitable mediators can include, but are not limited to, metallic nanoparticles, graphene, carbon nanorods, multi-walled or single-walled carbon nanotubes, and/or quantum dots (ultrafine particles with particle size of 1-10 nm). Suitable labels can include (but are not limited to) methylene blue, ferricyanide, gold, or any electroactive species known or used in the art. As set forth in detail herein below, after the sensor and working electrode come into contact with solution containing the antigen, the antigen binds to the surface-bound affinity probe or undergoes a chemical reaction with materials on the working electrode, resulting in a change to the surface impedance or voltammetric response or the production of an electroactive species. As a result, an amperometric or potentiometric signal is output, which can be measured.

For example, silver nanoparticles or copper oxide can be used to detect creatinine, nickel oxide to detect urea concentration, and cupric oxide can be used to detect fructose concentration. Other methods for fructose measurements can include LaMnO₃ nanofibers, Co₃O₄—CuO, Nano-Ni(II)-curcumin, and Ni-DPA. Ferricyanide, L-cystein-modified silver electrodes, lauryl gallate, hematein, and arsezano I can be used to detect albumin. Carbon-based materials show redox behavior in the presence of several vitamins and proteins, as well as various polymers, metals, ceramics, composites, and nanomaterial structures such as nanoparticles, graphene, quantum dots, nanotubes, nanowires, and various polymer encapsulation schemes. Dyes including alizarin red can be used for calcium detection.

In some embodiments, the working electrode can function as an ion selective electrode, using membranes, dyes, ionophores, chelators, aptamers, other affinity probes, chemicals, and/or any other materials that selectively sequester ions of interest (e.g., calcium, sodium, potassium, iodine, magnesium, selenium, manganese, zinc). Other materials can be incorporated or coated onto the working electrodes to reduce or eliminate non-specific binding and matrix effects. Suitable techniques can include the use of films, such as Nafion® (available from DuPont USA, Wilmington, Del.), surfactants (e.g., PEG), and/or proteins (e.g., BSA).

In some embodiments, the disclosed biosensing device can include electronics (such as a potentiostat) that enables electrochemical interrogation of the electrodes. The term “potentiostat” as used herein refers to an electronic instrument that controls the voltage difference between a working electrode and a reference electrode by injecting current into the system through a counter electrode. The electrochemical detection can be performed via amperometry (chemical analysis by techniques involving measuring electric currents), voltammetry (methods where the electrode potential is varied while the current is measured), potentiometry (methods of determining the potential between two electrodes), impedimetric (methods of measuring impedance or conductance), and/or any other known electrochemical technique. For example, in some embodiments, the detection can include linear voltammetry (a method of determining electrochemical properties wherein working electrode potential is ramped linearly versus time), cyclic voltammetry (a method of determining electrochemical properties wherein working electrode potential is ramped linearly versus time, and once reaching a set potential, the working electrode's potential ramp is inverted, and the current is measured between the working electrode and the counter electrode), staircase voltammetry (a method of determining electrochemical properties wherein working electrode potential is increased in a step-wise, pseudo-linear fashion), chronoamperometry (electrochemical technique in which the potential of the working electrode is stepped against a counter electrode or switched in from open circuit and the resulting current from faradaic processes occurring at the electrode is monitored as a function of time), chronocoulometry (the measurement of the charge of electroactive species adsorbed on to an electrode with respect to time), differential pulse voltammetry (technique in which a square wave pulse superimposed on a potential dc ramp is applied on the sensing electrode and the differential current output is plotted against the applied dc potential), and/or square wave voltammetry (technique where potential is swept not in a linear fashion but in a saw-tooth manner to minimize capacitive currents). Alternatively or in addition, electrochemical impedance spectroscopy (the study of the variation of the impedance of an electrochemical system with the frequency of a small-amplitude AC perturbation) can be used for quantification. Labels and/or mediators can be included in the design to promote more efficient electron transfer and signal amplification.

In some embodiments, the substrate onto which the electrode materials are deposited can be paper-based. Paper-based substrates provide an inexpensive material capable of permitting electrode printing. For example, screen-printing or ink-jet printing can deposit many different electrode materials, such as conductive carbon inks, carbon paste, carbon nanotubes, or various metals or other conductive materials. It should also be appreciated that the substrate can be constructed from a variety of other materials, such as (but not limited to) ceramics, polymeric materials (e.g., polyethylene terephthalate), and the like.

In some embodiments, the disclosed system can be multiplexed to simultaneously test for a wide variety of vitamins, minerals, and other dietary nutrients by incorporating electrodes for sensing biomarkers for each nutrient. As used herein, the term “multiplex” refers to simultaneously and/or separately conducting a plurality of assays on one or more multi-test platforms, such as simultaneously and/or separately using a plurality of working electrodes on a single biosensor platform to measure an array of biomarkers. For example, an antibody or aptamer developed for methylmalonic acid (MMA) can be used to detect clinical deficiency of vitamin B12 stores. The antibody or aptamer can be biotinylated and bound to the surface-bound streptavidin, or can be fixed in any way known or used in the art (i.e., covalent bonding). The working electrode can work in a similar way, performing electrochemical spectroscopy to determine the change in the electrochemical response to quantify the urinary biomarker. In this way, any number of working electrodes bound with various targets can be simultaneously or serially subjected to electrochemical spectroscopy to obtain a measurement and quantify concentration levels. A working electrode that specifically targets creatinine can be used in the multiplexed array in order to normalize the results from the other working electrodes.

Other techniques can also be used to detect key micronutrients. For example, EGTA selectively chelates calcium, such that a derivatized version of EGTA can be biotinylated (or covalently bound to an electrode surface) to detect and quantify urinary calcium levels. Particularly, biotinylated-derivatized EGTA saturates the streptavidin/avidin immobilized electrode surface and acts as a linker for detecting calcium using EIS. Biotinylation can be used to link any affinity probe to streptavidin, avidin, or other avidin-like derivative immobilized on the electrode surface. Further, in some embodiments, an aptamer developed for targeting a urinary biomarker (such as methylmalonic acid) can be biotinylated to act as a linker between the streptavidin-coated electrode surface and methylmalonic acid in solution.

A multiplexed sample can comprise any number of working electrodes, each targeting a separate and distinct urinary biomarker. For example, a single sensor design can comprise one reference electrode, one counter electrode, and one or more (e.g., 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more) working electrodes to detect one or more (e.g., 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more) urinary biomarkers. Thus, in some embodiments, a multiplexed biosensor can have 6-8 working electrodes, one counter electrode and one reference electrode. Surfactants can optionally be incorporated to improve production processes, such as Triton X or Tween for better controlling for undesirable surface tension effects. Further, meshes, channels, hydrophobic and/or hydrophilic coatings can be used to aid solution spreading and solution capture by controlling fluid absorption and capillary action via surface tension to better control sensor manufacturing and reproducibility as well as working solution uptake and volume control.

Thus, the working electrodes can be customized as needed to detect one or more desired urinary biomarkers. For example, the disclosed sensor can comprise at least one working electrode comprising copper (I) oxide incorporated into carbon ink at 0.5-10 weight percent (e.g., 2%) for creatine detection. Further, the sensor can comprise at least one working electrode comprising nickel oxide incorporated into carbon ink at 0.5-10 weight percent (e.g., 5%) for urea detection. Sodium hydroxide or potassium hydroxide can be added to the working electrode at 1-10M (e.g., about 5M) to control solution pH and improve reaction chemistry. Polymers can be used to immobilize the hydroxide-containing chemicals, such as poly(ethylene oxide), polyvinyl acetate, polyvinylpyrrolidone, or any other polymer or combination thereof. Also, channelized biosensor designs can be used to physically direct fluid flow via capillary forces for localized conditions suitable for electrochemical biomarker detection.

Continuing, in some embodiments, the disclosed sensor can comprise at least one working electrode comprising about 1-30U (e.g., 10-20U) fructose dehydrogenase (FDH), invertase (sucrase), 0-5 mM ferricyanide (e.g., 3 mM), and 0-2% nafion (e.g., 0.01%), used to detect fructose and sucrose. Alternatively, LaMnO₃ nanofibers can be used to detect fructose, particularly with similar pH control. In some embodiments, the sensor can comprise at least one working electrode that includes N8-acetylspermidine aptamer for detection of N8-acetylspermidine. The aptamers can include methylene blue conjugated to one end and a thiol conjugated to the other. The thiol-conjugated end can be immobilized on a gold working electrode or gold nanoparticle-coated working electrode. Other electroactive species, or other immobilization molecules can be used. In some embodiments, the sensor can comprise at least one working electrode that includes aptamer for putrescine, spermidine, and spermine for detection of putrescine, spermidine, and spermine (the aptamer binds specifically to all three). In some embodiments, an aptamer distinct for each of these three polyamines is used on either a single electrode or three distinct working electrodes. In some embodiments, the aptamers have methylene blue conjugated to one end and a thiol conjugated to the other. The thiol-conjugated end can be immobilized on a gold working electrode or gold nanoparticle coated working electrode. Other electroactive species, or other immobilization molecules can be used. In some embodiments, the sensor can comprise at least one working electrode that includes ferricyanide, L-Cystein-modified silver, hematein, lauryl gallate, or arsezano I for the detection of albumin.

Alternatively, in some embodiments, multiple distinct sensors can be used, each quantifying a distinct urinary biomarker. Further, it should be appreciated that other designs can be employed to accommodate the needs of a disposable or standalone electrochemical analyzer. For example, in some embodiments, the sensors can be built on a device that includes the electronic circuitry and antennas necessary for powering an AC signal to each electrode and wirelessly transmitting the data to a separate unit for analysis.

In some embodiments, a multiplexed biosensor can be read using an electrochemical analyzer with data transfer capabilities, such as (but not limited to) wireless transfer capabilities. For example, the disclosed device can comprise electronics capable of wireless data transmission via Bluetooth®, Bluetooth Low Energy®, Wi-Fi®, or any other mechanism for wireless data transfer. Wireless data transmission can provide the instructions for beginning a test, completing a test, adjusting the device electrodes or other mechanical components, operating motors, cleaning the device, storing data, and/or receiving test data. The data transmission can occur between the device and a smartphone, smartwatch, router, other sensors, and/or other electronic device. The data transmission can occur using a router or other wireless internet access and into cloud storage for processing and analysis.

In some embodiments, the electrochemical analyzer can be a standalone unit that accepts urine samples and sensors. Alternatively or in addition, the electrochemical analyzer can be a standalone unit that has been pre-filled with a testing solution and accepts sensors that have already been exposed to the urine sample to be tested. In some embodiments, the electrochemical analyzer can be designed to integrate with a disposable sensor unit as a completely disposable and one-time-use system. In some embodiments, the electrochemical analyzer can be a unit designed to attach to a toilet to test the diluted urine sample and/or the electrochemical analyzer and sensors can be integrated into the toilet, as set forth in more detail herein below.

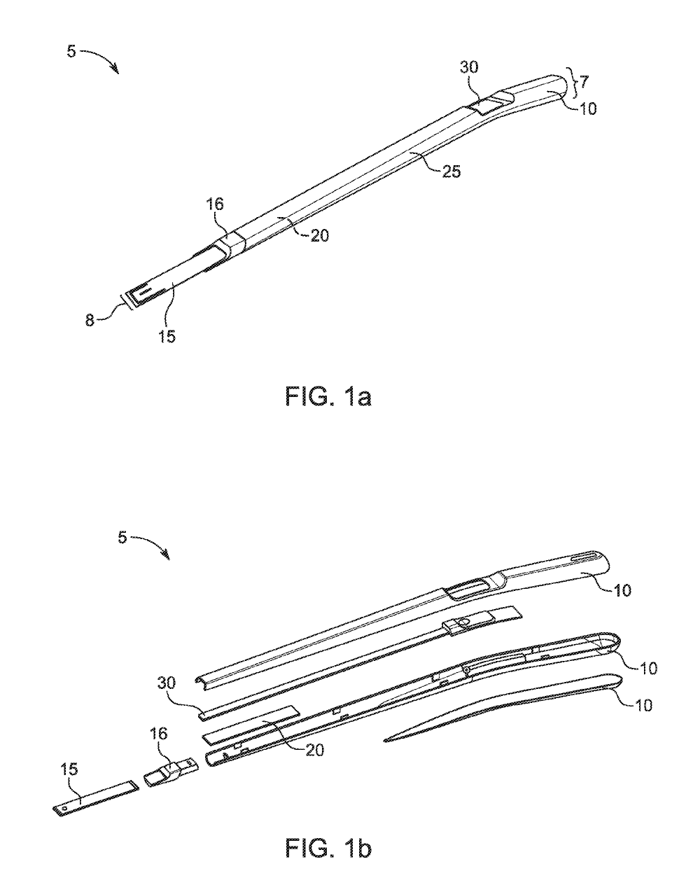

FIGS. 1a and 1b illustrate one embodiment of sensor 5 that can be configured for use with a toilet. Particularly, sensor 5 comprises first end 7 that includes handle 10. The device further includes second end 8 comprising biometric strip 15, strip connector 16, and electronics board 20 configured within the sensor interior. Board 20 comprises a multiplexed potentiostat and wireless data transfer hardware. The device optionally comprises ejection element 30 that can be selected, pushed, or adjusted to move, store, and/or eject biometric strip 15 and/or connector 16 from the second end of the sensor. The disclosed sensor can be used to detect urinary biomarkers that can be correlated to a subject's nutritional health and/or to a disease or disorder associated with the subject (e.g., kidney disease).

As set forth above, device 5 comprises handle 10 at first end 7 configured to provide a gripping element for the user to handle the device. For example, in some embodiments, the handle can be sized and shaped to correspond to a human hand. The handle can be configured in any desired cross-sectional shape, such as (but not limited to) round, rectangular, oval, conical, octagonal, or polygonal-shaped. The handle can include one or more gripping elements, such as raised ridges, textured areas, and the like to allow the user to properly maintain control of handle 10 during use. In some embodiments, handle 10 can include at least one surface with indentations that correspond to a user's hand. It should be appreciated that handle 10 is not limited and can include any design known or used in the art.

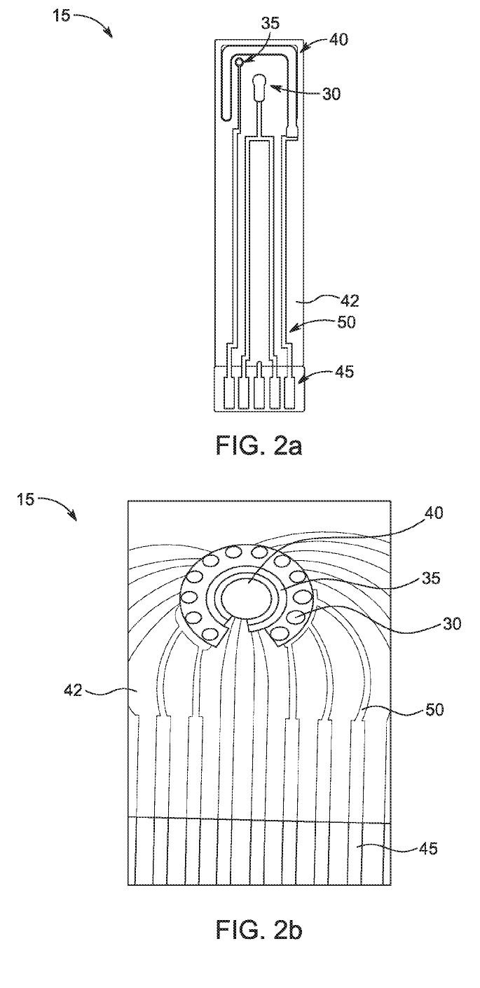

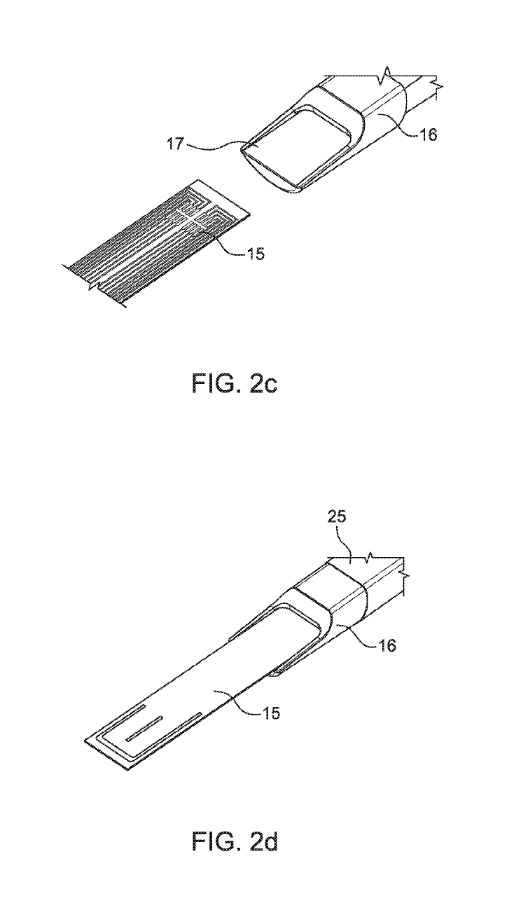

FIG. 2a illustrates one embodiment of biometric strip 15 that can be used with disclosed sensor 5. Particularly, strip 15 comprises at least two disposable electrodes for sensing. In some embodiments, strip 15 can be used for a single detection of urinary biomarkers in one sample of fluid, after which the strip is disposed of. In some embodiments, strip 15 can be used for more than one detection event for urinary biomarkers in more than one sample of fluid, after which the strip is disposed of. Strip 15 can comprise working electrode 30, reference electrode 35, and counter electrode 40 configured on inert substrate 42. Alternatively, strip 15 can comprise a working electrode and a combination reference and counter electrode. It should be appreciated the biometric strip of FIG. 2a is not limited and the electrodes can be configured in any desired orientation. Substrate 42 can be constructed from a wide variety of materials known and used in the art, including (but not limited to) paper, polymeric materials, ceramic materials, and the like. Further, the disclosed substrate can be configured in any desired shape, such as square, rectangular, circular, oval, abstract, and the like. Screen printed biosensor strips are well known in the art. See, for example, U.S. Pat. No. 6,783,645, the entire content of which is hereby incorporated by reference herein.

Working electrode 30 is designed to selectively detect and quantify various biomarkers in biological fluids, such as urine, diluted samples comprising fecal matter, and/or other materials. The working electrode can incorporate chemical, enzyme, and/or any other affinity probe-based method (i.e., aptamer, antibody, etc.) of selective electrochemical detection. The chemicals, mediators, enzymes, aptamers, antibodies, and/or other elements can be incorporated and mixed into the electrode material itself, or can be applied to the surface of the electrode via drop-coating, physical absorption, covalent binding, ionic binding, hydrophobic interactions, biotinylation, cross-linking, entrapment, electrodeposition, and/or any other surface immobilization technique. Such methods are well known to those of ordinary skill in the art.

In some embodiments, each working electrode is designed to detect and quantify a single biomarker. Alternatively, a single working electrode can be designed to detect and quantify multiple biomarkers simultaneously, as shown in the embodiment of FIG. 2b, illustrating strip 15 comprising a plurality of working electrodes 30. Thus, in some embodiments, electronics board 20 allows multiplexing, which enables the electrochemical interrogation and processing of multiple working electrodes (any number greater than one). Alternatively, the presently disclosed subject matter also includes embodiments wherein electronics board 20 only allows one working electrode to be interrogated. The multiplexing can occur serially or in parallel.

In some embodiments, electrodes 30, 35, and 40 can be “two-dimensional” electrodes on paper or polymer-based substrate 42. The electrodes can be ink-jet printed, screen-printed, and/or deposited by any known method to produce relatively thin electrode structures in the nanometer to millimeter size range. Such methods are well known in the art. Electrodes 30, 35, and 40 can be configured in any desired shape. For example, the electrodes can be configured as circular, oval, or rectangular in shape in some embodiments. The electrodes can vary in circumference and length, but can be microscopic (dimensions of less than 10 micrometers) or macroscopic (dimensions of greater than 10 micrometers) in size.

Further, electrodes 30, 35, and 40 can be constructed from carbon, carbon paste, pyrolytic carbon, glassy carbon, silver, silver-silver chloride, potassium chloride, platinum, gold, nickel, copper, and/or any other conductive material or composite material known or used in the art. In some embodiments, the electrodes can comprise one or more chemicals, materials, and/or mediators infused within the electrode, such as (but not limited to) silver nanoparticles, copper nanoparticles, copper oxide nanoparticles, gold nanoparticles, nanofibers, graphene, graphene oxide, reduced graphene oxide, single-walled carbon nanotubes, multi-walled carbon nanotubes, zinc oxide nanoparticles, nickel oxide nanoparticles, polyaniline nanoparticles, and/or any other mediator material known or used in the art. Alternatively or in addition, such materials can be attached to the surface of the electrodes using any method, including (but not limited to) covalent bonding, physical adsorption, hydrophobic interactions, ionic bonding, entrapment, cross-linking, self-assembled monolayers, and the like.

As shown in FIG. 2a, strip 15 comprises electrical contact pads 45 that mate with electrical contacts on the housing that connect to a potentiostat and enable the electrochemical testing to be performed. Pads 45 can be constructed from conductive materials (such as those used for the electrodes). The disclosed strip further comprises connecting elements 50 that connect the electrodes and the contact pads. The connecting elements can be constructed from wires or lines made from the same or similar conductive materials as used in the electrodes and contact pads.

In some embodiments, biosensor strip 15 can cooperate with connector 16 as shown in FIG. 2c. Particularly, the connector includes recess 17 sized and shaped to allow the insertion of one end of the strip. The connector therefore releasably joins the biosensor strip to the remainder of the sensor device, as shown in FIG. 2d.

In some embodiments, sensor 5 can include a mechanism for expanding and/or contracting the length of the device as needed by the user (i.e., to make it easier to dip into the toilet). Such mechanisms are well known in the art.

In some embodiments, the disclosed method of electrochemical detection can include voltammetry. Specifically, the method can include the following parameters:

Linear Sweep Voltammetry: Voltage: −1<V<1.5 or any range in between, Rate: 1<mV/sec<1000.

Staircase Voltammetry: Voltage: −1<V<1.5 or any range in between, Step height: 0.001<V<0.1, Rate: 1<mV/sec<1000.

Cyclic Voltammetry: Voltage: −1<V<1.5 or any range in-between, Rate: 1<mV/sec<1000.

Chronoamperometry: Voltage: −1<V<1.5, Time: 1<sec<300.

Square-wave voltammetry signal: Voltage: −1<V<1.5, Frequency: 5<Hz<200, Amplitude: 1<mV<50, Incremental step height: 1<mV<10, The shape of the square pulse is symmetrical.

The current can be switchable in series or parallel (or an equivalently fast scheme). In some embodiments, the method can include several samplings at the end of each square wave form for each working electrode interrogated with a single stimulus signal. In some embodiments, the method can comprise a distinct stimulus signal with distinct samplings for each electrode run in parallel or serially. Optionally, an interposer can connect the board (potentiostat) to the sensor strips. Particularly, three or greater electrode connections using conductive connections can be configured between the potentiostat and the sensors having a unique conductive path for each.

Advantageously, sensor 5 can be designed to work in a continuous format without the need for sensor replacement after every cycle. Thus, the sensor can be configured to work indefinitely or for a fixed number of cycles before replacing any parts, including electrodes, electronics, batteries, fixation components, and the like.

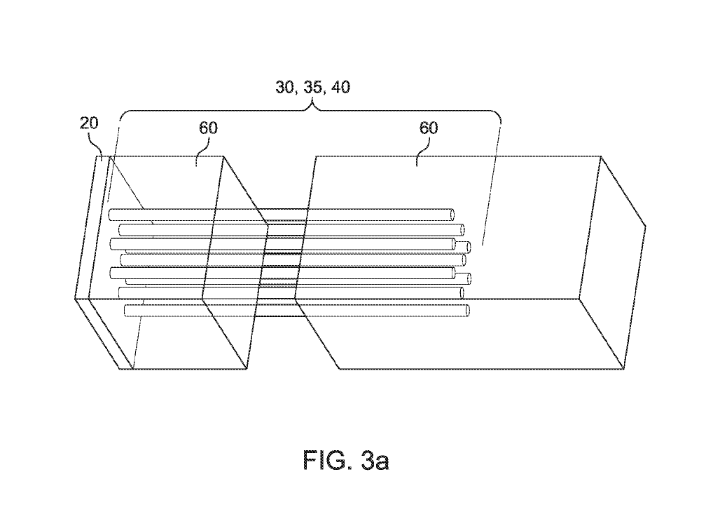

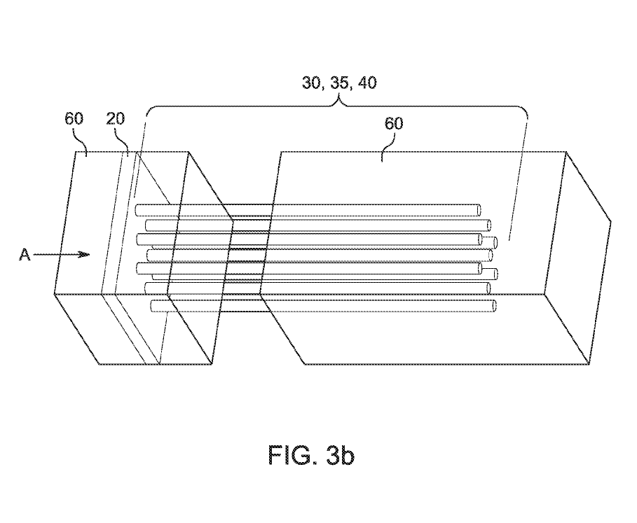

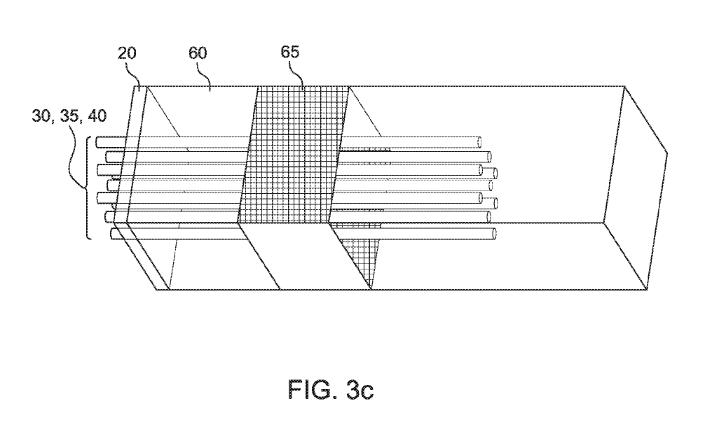

In some embodiments, the surfaces of the electrodes can be configured to be exposed to test solution (water containing biological fluids like urine and/or fecal matter) only within a fixed longitudinal gap, as shown in the sensor of FIGS. 3a and 3b. Particularly, electrodes 30, 35, and 40 can be connected to electronics board 20 configured within internal housing 60. The internal housing moves longitudinally after each testing cycle or after a fixed number of test cycles, as indicated by Arrow A in FIG. 3b. The longitudinal movement exposes a fresh surface area of the electrodes after each test or after a fixed number of tests. The exposed surface area of the electrodes may or may not be fixed. If not at a fixed length, the surface area can be determined based on the amount of standing water in the toilet or urinal by manually or electronically adjusting the height of sensor 5, such as through the use of software or automatically by an external sensor. The sensor controls the amount of exposed surface area and thus the amount of potential biomarker binding.

Movement of the electronics, electrodes, housing, and/or gap height can be performed by any mechanical mechanism capable of incremental movements, such as (but not limited to) the use of servomotors and stepper motors. In some embodiments, movement can be triggered wirelessly via a smartphone, smartwatch, computer, and/or other device. Alternatively or in addition, the mechanisms can be triggered by sensors and/or the detection of one or more trigger activities (e.g., flushing a toilet, sitting, rising from a sitting position, and the like). After a new, fresh electrode surface is exposed post-extension, the previously exposed portion of electrodes 30, 35, 40 enters housing 60, thereby hiding the used surface from exposure to new fluids, as shown in FIG. 3b. A gasket or other mechanism can shield hidden portions of the electrode from the biological fluids and/or water.

In some embodiments, housing 60 comprises a single unit that houses all of the electrodes, as shown in FIGS. 3a and 3b. However, the presently disclosed subject matter also includes embodiments wherein the housing comprises individual sheathing over each electrode.

Although depicted as cylindrical in shape in FIGS. 3a and 3b, the presently disclosed subject matter also includes embodiments wherein electrodes 30, 35, and/or 40 are flat. Particularly, a flat design can be advantageous in embodiments wherein one or all electrode faces are exposed to the water and/or biological fluid.

Biological fluids and/or water containing biological fluids (including urine and/or fecal matter) have physical access only to the exposed electrode surfaces in the sensor of FIGS. 3a and 3b. After each test and toilet flush, the fluids are removed before the next use. In some embodiments, the fluids can be naturally removed along with normal flushing, having open access points to the surrounding fluid. In other embodiments, the fluid can be brought into the internal device housing via pumps or valves and released upon test completion. In some embodiments, device 5 includes blocking element 65 (e.g., mesh, membrane, and/or other blocking structures) that permit only fluids (no solids) to enter the internal housing and exposure to electrode surfaces, as shown in FIG. 3c. In this way, solid material is prevented from contacting and fouling the electrode surfaces.

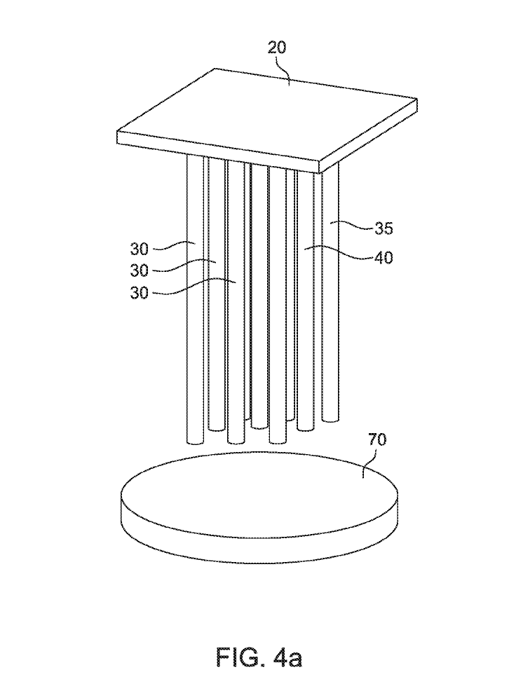

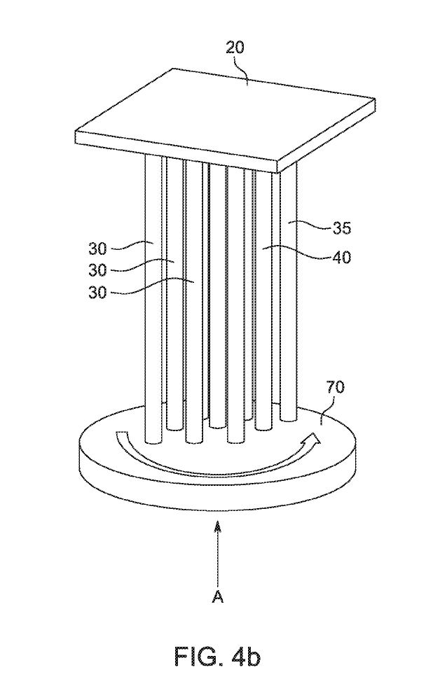

In some embodiments, each electrode surface can be mechanically abraded to remove a thin layer of material, as shown in FIGS. 4a and 4b. Particularly, abrasive element 70 contacts the surface of electrodes 30, 35, 40 to mechanically remove a thin layer of the electrode, thereby exposing a fresh electrode surface. The abrasive element is not limited and can include any rotary, uni-axial, or other element capable of dimensionally-oriented abrasive motion. Abrasive element 70 can comprise any material capable of removing a thin layer of electrodes 30, 35, and 40 with adequate sensing capabilities intact. For example, in some embodiments, the abrasive element can comprise an alumina-based or silica-based sandpaper-like material with a fine surface roughness. In some embodiments, water from the toilet can be used to clean the electrode surface. Alternatively or in addition, a cleaning solution can be dispensed from the device to clean the electrode surface.

In some embodiments, each electrode surface can be chemically treated to remove a surface layer. The chemical etches the electrode surface to remove a thin surface layer to thereby expose a fresh electrode surface. Alternatively or in addition, the chemical can cause any bound surface molecules to detach from the electrode surface or any chemical binding. For example, EDTA with formamide can be used to elute streptavidin-biotin interactions. Further, in some embodiments, the surface of each electrode can undergo an electrochemical cleaning by applying a continuous or variable voltage one or more times. The electrochemical treatment causes any bound molecules to detach or un-bind, and renders the electrode surface in a fresh-like state such that surface binding can once again occur.

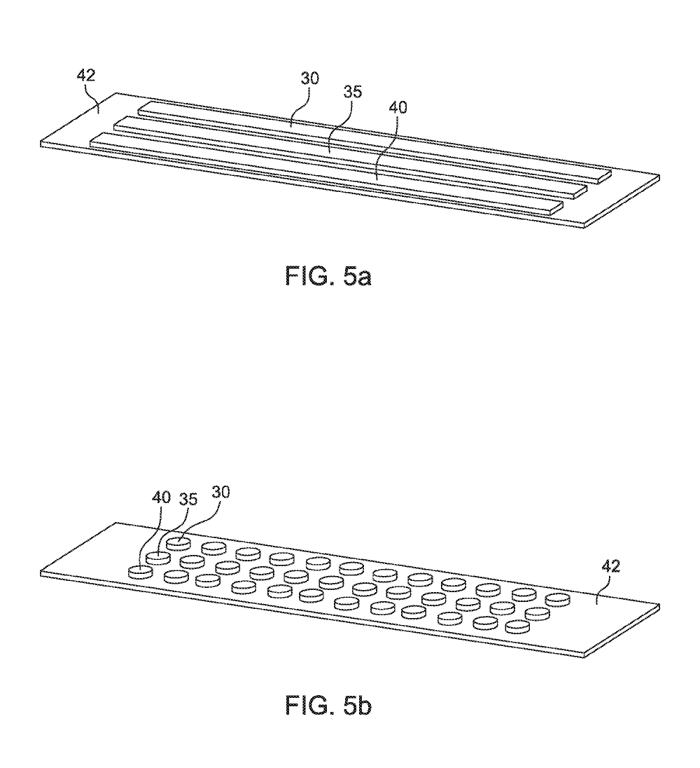

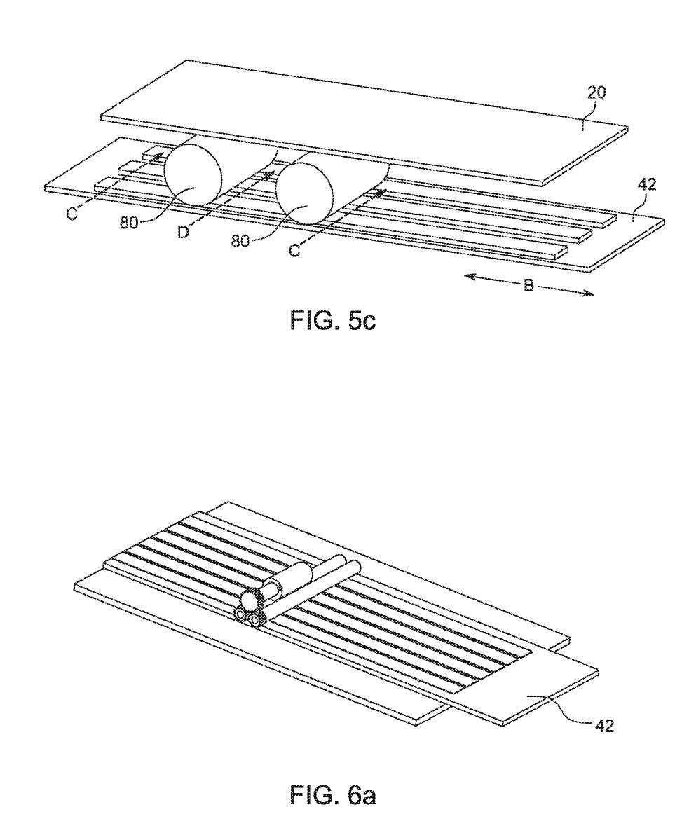

In some embodiments, the electrodes are inkjet-printed, screen-printed, or deposited using any other known form of two-dimensional deposition onto paper or polymer-based substrate 42. The electrodes can be configured in any desired shape, such as an elongated rectangular shape or with repeated, distinct electrodes, as illustrated in FIGS. 5a and 5b, respectively. As shown in FIG. 5c, substrate 42 and/or housing 60 can be moved to expose new regions of the electrodes before, during, or after testing. In embodiments wherein the electrodes are repeated in distinct units, they can be connected and can share a similar reference and counter electrode. In another embodiment, each set of working electrodes can include distinct counter and reference electrodes, where a mechanism can change the electrode connections between substrate 42 and electronic hardware 20 after each movement of the substrate. A similar mechanism for moving the substrate or the housing to connect to the new electrodes can be employed during testing, as shown in FIG. 5c. Particularly, electronic hardware 20 is coupled to substrate 42 such that the electrodes are connected but the substrate can move longitudinally, as shown by Arrow B. In some embodiments, cylindrical rollers 80 can be used to produce movement. The housing can be designed such that fluid cannot pass through regions C, but can pass through region D.

In some embodiments, the screen-printed electrodes only include working electrodes 30. In such embodiments, reference electrodes 35 and counter electrodes 40 can be positioned on separate screen-printed electrodes or can be configured as solid electrodes. The counter and reference electrodes can be configured to be stationary within the fluid-accessible region. Alternatively, the counter and reference electrodes can be configured as separate elements but still continue to move with the working electrode to expose fresh electrode surfaces.

In some embodiments, the mechanism for moving substrate 25 involves cylindrical rollers 80. The substrate can traverse a single plane, or it can bend and/or roll into another plane during movement. The substrate can be connected to electronic hardware 20 once during installation and the same connection holds during any movement of the substrate or housing. Alternatively, the electrode connections to the electronic hardware can switch during movement. In some embodiments, the substrate can be replaceable.

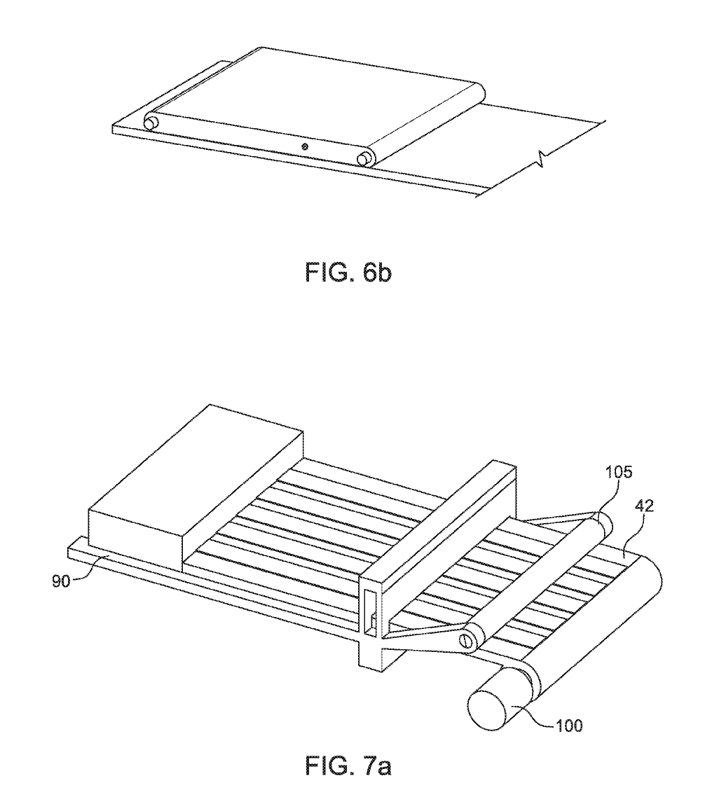

FIG. 6a illustrates one embodiment of a sensor strip movement mechanism using rollers controlled by a micro-stepper motor and gears. FIG. 6b shows a roller mechanism that covers electrode regions not in use.

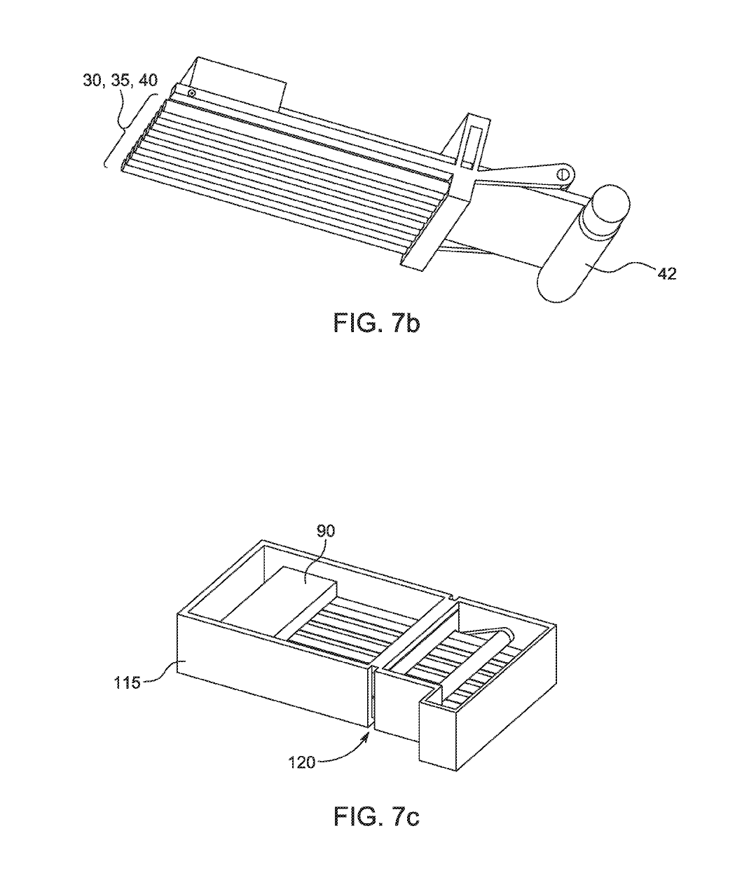

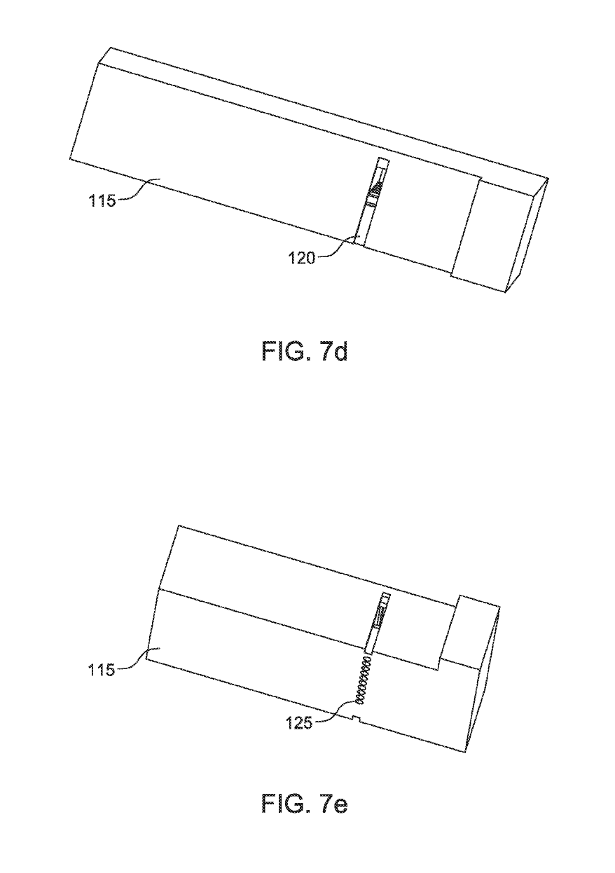

FIGS. 7a-7e illustrate various embodiments wherein the substrate comprising the electrodes is moved incrementally using a gear, rotor, or other rotation element attached to the distal end of the substrate via chemical or physical attachment. Upon each rotation, substrate 42 is pulled towards rotation element 100. The substrate wraps around the rotation element during repeated operation. FIG. 7a illustrates substrate 42 attached to rotation apparatus 90 using any method known in the art and configured to wrap around rotation element 100 (e.g., pin) during incremental movements to drive the substrate in a longitudinal direction. Support element 105 can be used to keep the substrate in place and to prevent excess bending. FIG. 7b illustrates wires connecting the working electrodes, reference electrodes, counter electrodes, and motors to a central housing that permits the electrodes to remain in electrical contact while the substrate moves.

FIG. 7c-7e illustrate optional housing 115 that encloses rotation apparatus 90 to only permit fluid to flow through channel 120 to access a portion of the substrate to perform an electrical test before an incremental strip movement. Housing 115 can include electrical contact access points 125 to connect with the electronic board, such as (but not limited to) potentiostat hardware, wireless signal transfer electronics, batteries, and the like, as shown in FIG. 7e. Housing 115 can be constructed from any of a variety of materials known or used in the art, including (but not limited to) ceramics or polymeric materials. Further, housing 115 can be configured in any desired shape.

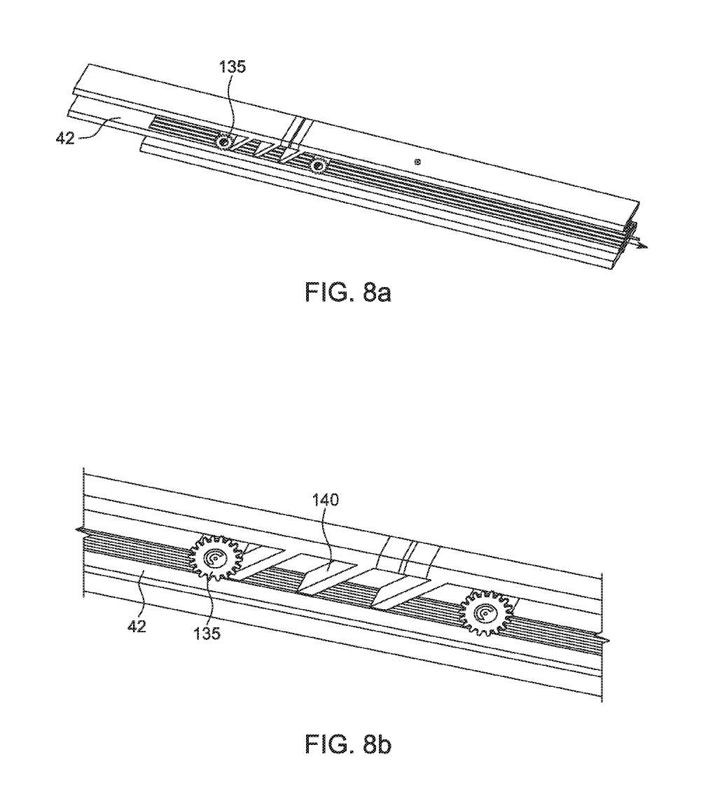

In some embodiments, one or more valves can be used to prevent water from accessing unintended electrode surfaces. The valves can seal backflow using pressure (e.g., spring-loaded pressure). The valves allow the substrate or housing to move for additional testing either by forcing the substrate through at greater pressure than the valves can withstand, or using a gear or other mechanism to open the valve momentarily during substrate movement. In some embodiments, flaps 130 can used to prevent water backflow, as shown in FIGS. 8a and 8b.

Particularly, substrate 42 moves through the housing 115 via at least one gear-controlled roller 135 incrementally during each testing period. FIG. 8b illustrates a magnified testing region illustrating valves 140 that can be spring-loaded, fixed, and/or controlled by any other mechanism to prevent fluid backflow. In these embodiments, fluid can only access the internal region between two of the valves (a third valve could be implemented as a backup). In this example, permanent reference and counter electrodes can be included that do not move with the sensor strip.

Flaps 130 can rest upon the housing surface, substrate 25, rotation element 90, or other components separating the fluid-filled region and any dry regions inside the housing. In some embodiments, gaskets, rollers, and/or wheels can prevent water backflow. The flaps (as well as any valves) can be constructed from hydrophobic materials or can be surface treated to render them hydrophobic. These elements can also be designed using materials or surface treatments to prevent biofouling. The remainder of the of the internal housing components can be similarly treated to prevent accidental damage, but these regions should remain dry. The external housing 115 can be made using any material known or used in the art, such as (but not limited to) polymeric materials, wood, and/or metals. In some embodiments, the external covering can include surface paints and/or additives that render the materials anti-bacterial. Such paints and additives are well known in the art.

In some embodiments, each sensor strip comprises only the electrodes and geometries necessary to perform a single test. For example, the sensor strip substrate can be made from a biodegradable paper substrate, enabling the sensors to be easily ejected and flushed down the toilet. In some embodiments, the housing can include a storage compartment for multiple testing substrates. Internal mechanisms enable a new, unused test substrate to extend from the housing and contact liquid within a toilet or urinal that may comprise biological fluids. A test can be initiated by a user and/or automatically triggered. After the test is complete, the strip is ejected into the water and flushed.

In some embodiments, the sensor strip does not eject from the housing but is instead stored within the housing for disposal at a later time. The housing can enclose more than one test strip, and internal mechanisms that enable multiple tests before a user needs to physically manipulate the housing. The housing can include storage for unused test strips and used test strips. Internal mechanisms move a new, unused test strip to enable its contact with liquid within a toilet or urinal that may contain biological fluids to perform a test. The strip can be retracted and stored in a separate, internal storage compartment, or within the same storage compartment. Suitable mechanisms can include rollers, gears, belts, and/or other elements for moving the strips into position.

In some embodiments, housing 115 can be designed to enable permanent or semi-permanent adhesion within a toilet or urinal. For example, the housing can enable permanent or semi-permanent adhesion of part of the housing, while a compartment of sensors can be replaced either manually or with a wand-like tool. The testing and strip manipulation can be actuated wirelessly through a connected device, or can be triggered automatically by sensing other external triggers (e.g., wireless signal strength, IR sensors for temperature sensing, color sensors, and/or timing, other sensors on or near the toilet or urinal, or behaviors from the user like standing or sitting).

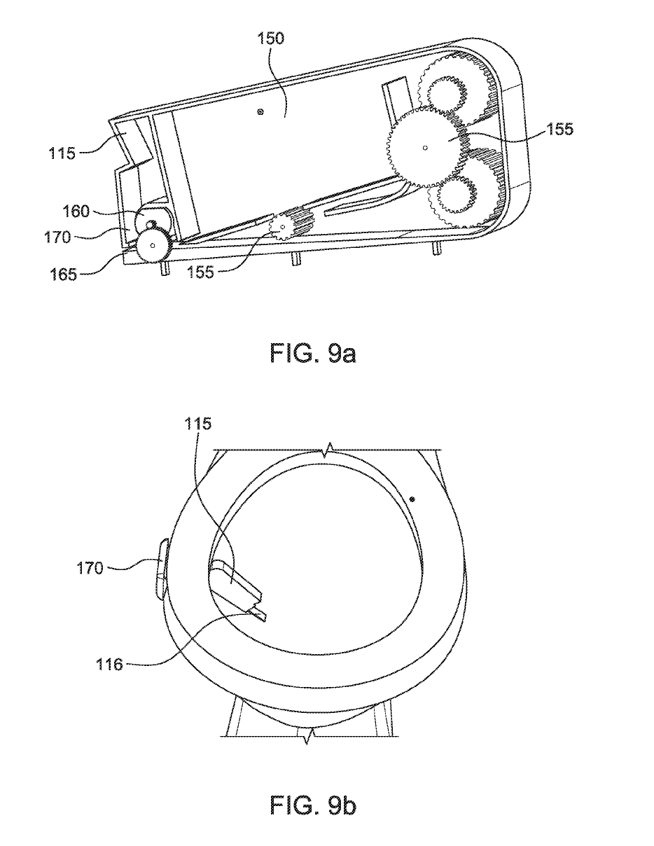

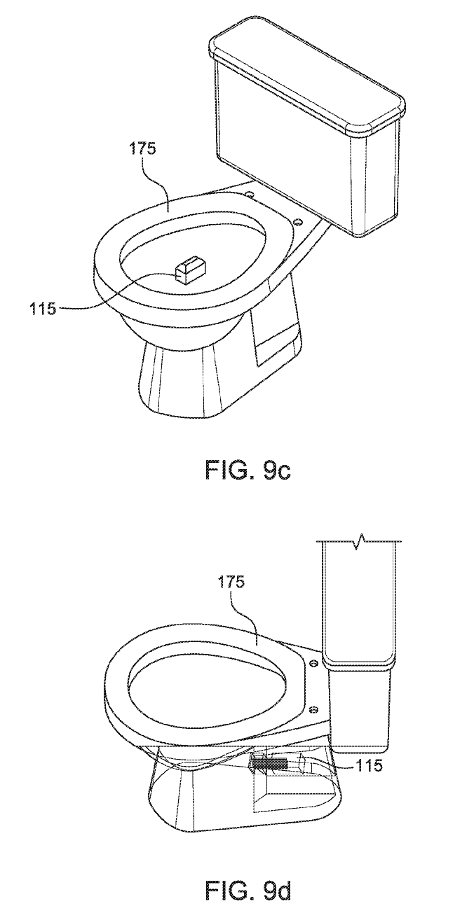

FIGS. 9a-9b illustrate one embodiment of an in-toilet design that houses disposable strips. Particularly, the design includes chamber 150 that houses at least one disposable sensor strip 116. The design further includes gears 155 and servo motors 160 that drive belts and wheels to translate a single disposable strip from chamber 150 through external opening 165. The strip extends until electrical contacts 170 (or timing-based, etc.) stop the motors with the sensor partly extended to enable submersion into the toilet water comprising the biological fluids. Electrical contacts 170 connect to contact pads on the disposable sensor strips and perform the electrochemical testing while the distal sensor end is submerged in solution. The electrical contacts are connected to a potentiostat that can be housed with the sensor storage and translation mechanism or external to the system, as demonstrated in FIG. 9b. An external sensor (such as an IR sensor) can be located on housing 115 to identify temperature changes in the fluid to alert the system to begin a testing sequence. Color sensors can also be attached to monitor changes in the environment and dictate the testing cycle. Gears 155 and motors 160 can operate in reverse once the test is complete to translate the disposable sensor back through to re-enter the storage compartment. In some embodiments, the storage compartment can be completely replaceable, although the presently disclosed subject matter also includes embodiments wherein the storage compartment is not replaceable.

In some embodiments, the device components can be integrated into a toilet seat. In some embodiments, the sensor device can be attached to the inside of toilet 175 (or urinal), as shown in FIG. 9c. The device can be attached using any mechanism, such as through the use of mechanical attachments (e.g., suction cups, screws, clips, fasteners) or an adhesive that enables the device to attach temporarily (until the user desires to remove it) to the inside of the toilet or urinal either below or above the water line. In some embodiments, the device components are integrated into the toilet or urinal manufacture and design, as shown in FIG. 9d. In some embodiments, the device is configured to allow the replacement of electrodes and/or batteries.

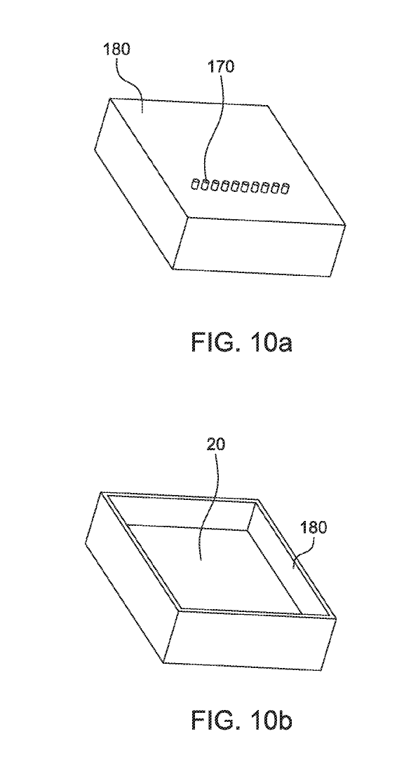

In some embodiments, housing 115 can be designed to be removed and replaced periodically. Alternatively, the housing can be designed to remain in position for an extended period of use, including sensor replacement. Advantageously, the housing can be designed to enable the removal and replacement of sensors without requiring removal of the housing unit from position. For example, FIGS. 10a and 10b illustrate one embodiment of cartridge 180 comprising electrical element 20 with contacts 170 that extend through the housing.

In some embodiments, the system can include a standalone cartridge 180 that houses each disposable sensor. The cartridges can be replaced manually or mechanically (e.g., through the use of a tool that assists with cartridge ejection and replacement). Cartridge 180 can comprise (in addition to the disposable sensor) one or more housing components, electronics, interposers, motors, gears, valves, and/or any other component. In some embodiments, the electronics, certain components of the housing unit and internal components, motors, and outer suction or adhesive agents, can remain in place during sensor replacement.

In some embodiments, a tool may accompany this process. The tool and all housing components including replaceable and nonreplaceable parts contain mechanisms for attaching and detaching to each other. For example, an extension or wand can include a clasp or locking mechanism to grip the sensor and/or cartridge for replacement. The device can include a manual or electronic locking and removal mechanism with the replaceable component, including (but not limited to) twisting about a screw-like connector, breaking a chemical or physical bond, unleashing a clasp or fastener, using an electronically controlled valve or any other locking mechanism. The wand can comprise the capability to replace the cartridge, the sensor, or the entire housing unit controlled by mechanical levers or buttons or electronically controlled locking mechanisms. The disposable sensor or cartridge, upon replacement, connects electronically to the electronic hardware in embodiments where the electronic hardware remains in place. Electronic connections can be made using mating ends suitable for underwater connections. Other connections may exist between the permanent unit and the disposable cartridge, including (but not limited to) connections to servo motors or stepper motors, wires, or other electrical, mechanical, or chemical components. A tool for replacing a sensor or cartridge may remove the used sensor and replace with a fresh sensor or cartridge in one procedure, or it may take multiple procedures.

Other functionality can be incorporated into the disclosed sensor. For example, toilet-bowl cleaning solution may be added to the device to provide multiple functionality (e.g., biomarker tracking plus cleaning).

Data can be stored on the biosensor device for any length of time. Data can be transferred to a back-end data application for storage and analysis. In some embodiments, the signal data can be analyzed to quantify urinary biomarker levels, and may be normalized against creatinine levels. If desired, the data can be presented and/or displayed to notify a user of macronutrient levels, micronutrient levels, and/or other dietary intake patterns. In some embodiments, the data can be compared against recommended daily allowances and correlations developed and known between urinary biomarker levels and dietary intake. In some embodiments, the data can be compared to national averages or averages among cohorts within a proprietary database. The comparisons can be used to suggest dietary changes and/or recommend specific supplement use.

Thus, in some embodiments, the disclosed biosensor device can be designed to temporarily interact with, attach to, and/or integrate with a toilet or urinal for biomarker detection and quantification in biological fluids, specifically urine and/or fecal matter. The sensor employs electrochemical detection mechanisms with a multiplexed array of working electrodes. The device houses a potentiostat with wireless data transmission capabilities, and a mechanism for enabling multiple uses. Access to a user's personal health data can be used to improve health and wellness outcomes, such as diet and nutrition, nutritional therapy, medical diagnosis, and/or monitoring disease progression.

Data can be transferred to a back-end data application for storage and analysis. In some embodiments, the electrochemical spectroscopy data can be analyzed to quantify urinary biomarker levels, potentially normalized against creatinine levels and/or osmolality. If desired, the data can be presented and/or displayed to notify a user of micronutrient levels. In some embodiments, the data can be compared against recommended daily allowances and correlations developed and known between urinary biomarker levels and dietary intake. The comparisons can be used to suggest dietary changes and/or recommend specific supplement use.

In some embodiments, the data can be used to design custom nutritional supplements. For example, the supplements can comprise varying ratios of vitamins, minerals, and/or other nutrients that specifically depend on the quantified biosensor data collected from a subject's urine sample. Nutritional supplements can be produced using any method known or used in the art. For example, in some embodiments, 3D printing can be used to create consumable pharmaceutical tablets with varying concentrations of multiple drugs, such as those being developed by Multiply Labs) (Goyanes, A. et al. 3D Printing of Medicines: Engineering Novel Oral Devices with Unique Design and Drug Release Characteristics. Mol. Pharm. 12, 4077-4084 (2015); Goyanes, A., Buanz, A. B. M., Hatton, G. B., Gaisford, S. & Basit, A. W. 3D printing of modified-release aminosalicylate (4-ASA and 5-ASA) tablets. Eur. J. Pharm. Biopharm. 89, 157-162 (2015); Khaled, S. A., Burley, J. C., Alexander, M. R., Yang, J. & Roberts, C. J. 3D printing of tablets containing multiple drugs with defined release profiles. Int. J. Pharm. 494, 643-650 (2015); Goyanes, A., Buanz, A. B. M., Basit, A. W. & Gaisford, S. Fused-filament 3D printing (3DP) for fabrication of tablets. Int. J. Pharm. 476, 88-92 (2014)). Alternatively or in addition, supplements can be constructed using traditional powder-pressing methods with quantities dictated by the sensor data. In some embodiments, supplement-loaded filaments can be printed in ratios determined by each individual's dynamic needs, determined using the urinary biosensors.

In some embodiments, the disclosed device can be used to diagnose a disorder, such as chronic kidney disease. For example, chronic kidney disease can be diagnosed through the detection of urinary albumin and urinary creatinine. Albumin normally circulates in the blood. In patients with declining renal function, albumin passes through the kidney and gets excreted through urine. As renal function continues to decline, more albumin passes through urine. This condition (albuminuria) is, therefore, a clinical marker for chronic kidney disease. A more accurate way of assessing this condition is to assess the albumin-to-creatinine ratio, whereby both urinary metabolites are quantified and compared to normalize results. The disclosed device can be provided to at-risk patients via physician referral, and it would create a technologically superior product capable of accurately diagnosing CKD and monitoring disease progress all while reducing healthcare expenditures. Because wireless data transfer is now ubiquitous, the patient's caregiver can be allowed access to their patients' data remotely and instantly. As albumin-to-creatine ratios rise or fall, the patient and physician can be provided with real-time physiological data to support early and evidenced-based decision-making regarding the effectiveness of intervention strategies and planning future procedures. The addition of dietary sodium, potassium, phosphorus, and protein tracking can provide further value to the patient and caregiver for managing chronic kidney disease from home.

An at-home electrochemical biosensor, as described herein, would constitute a significant advancement in diagnostic and management technologies. Particularly, the device allows at-home testing to enhance testing chronicity and thus, improve diagnostic accuracy. In addition, accuracy of the sensors (e.g., low-level albumin-to-creatinine quantification) will increase detection of early-stage diseases and disorders (e.g., chronic kidney disease). Further, increased quantitative testing frequency can enable persistent monitoring of disease progression. In some embodiments, monitoring dietary protein and sodium intake can help patients better regulate nutritional therapy for disease and disorders.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

Vitamin B7 Quantification in Diluted Urine Samples using EIS A Metrohm Autolab III potentiostat with an FRA module (available from Metrohm AG, Herisau, Switzerland) was used to perform EIS measurements on DropSens 1110-STR dual screen-printed electrodes coated with streptavidin (available from DropSens, Ltd., Spain). An AC sine wave signal at 40 mV was used during a frequency sweep from 10⁵ Hz to 1 mHz at 5 points per decade. The tests were performed in deionized water, tap water, tap water containing 50 mg NaCl, phosphate buffered saline (PBS), and urine samples. In addition to pure urine samples, urine samples were tested at 1:10, 1:100, and 1:1000 dilutions with water. Prior to testing the urine samples, some sample electrodes were blocked using 3 wt % BSA in PBS. Between each test, the samples were washed in the buffer solution and allowed to rest in the buffer for 10 minutes prior to testing.

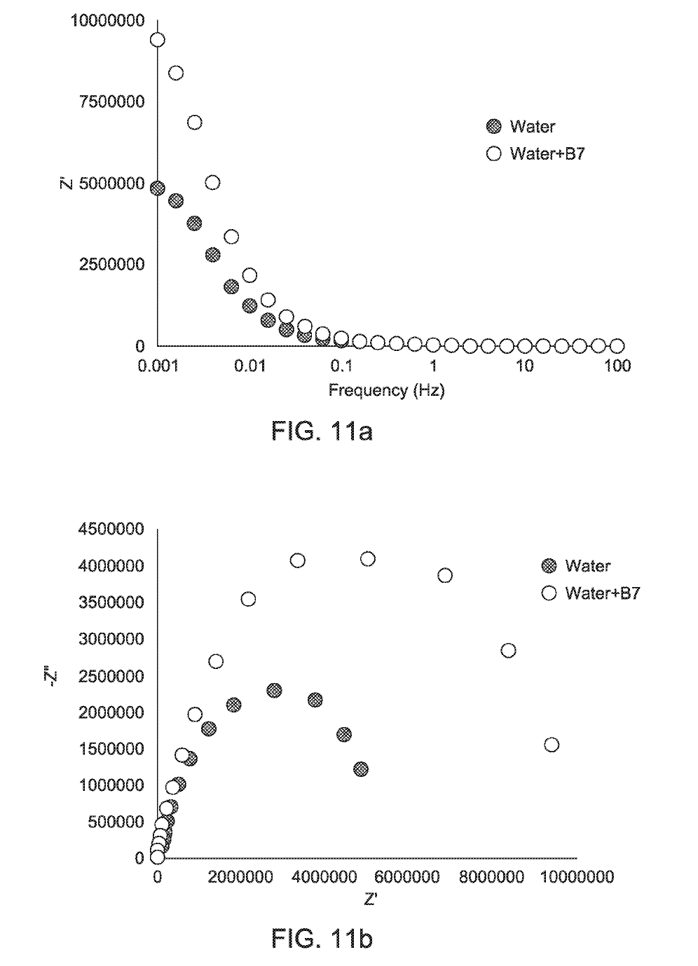

Cyclic voltametry measurements were performed using a sweep from −1 to 1 V. Lissajous plots were performed to verify linearity, resolution plots were performed to verify the sensitivity of the system, and Nyquist and Bode plots were used to analyze the surface impedance and quantify vitamin B7 (biotin) concentration. EIS spectra was taken before and after exposure to samples with vitamin B7 (biotin). The data is shown in FIGS. 11a and 11b, indicating clear changes to the Bode and Nyquist plots associated with an increase in impedance upon binding of the biotin to the streptavidin-coated electrode surfaces. The increase in charge transfer resistance occurred in the low-frequency regime associated with interface phenomena, whereas the solution resistance was associated with high-frequency regime and was essentially equal in both samples.

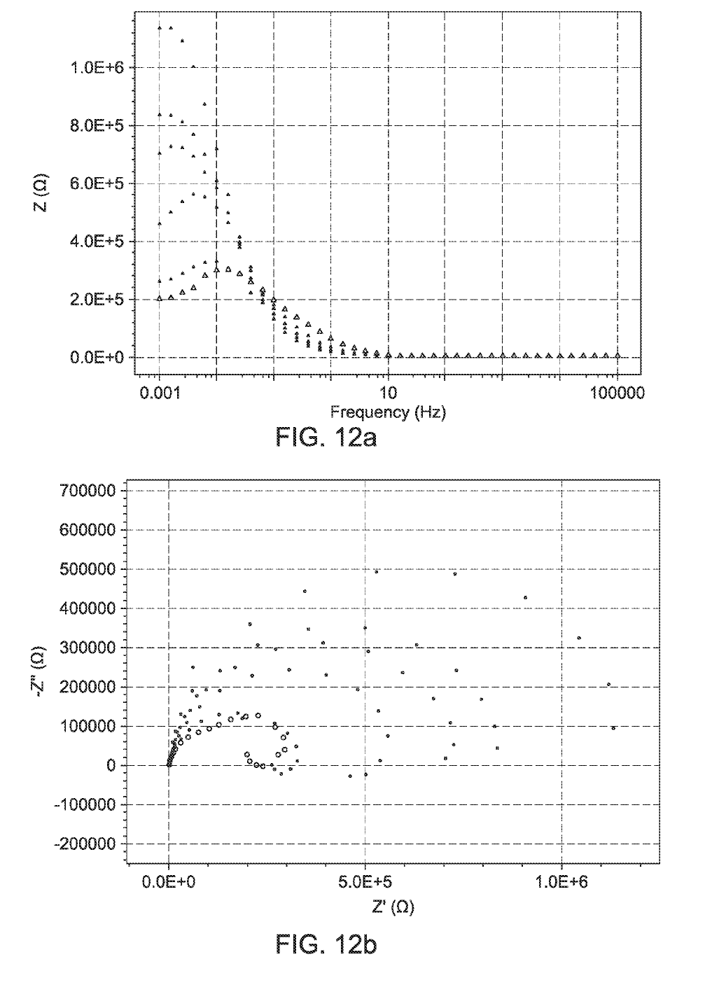

Urine was diluted 1:100 with water and was spiked with 50 nmol of biotin. 3 μl of the sample was spot dried on the electrodes before testing in tap water containing 50 mg NaCl. FIGS. 12a and 12b illustrate Bode plots and Nyquist plots, respectively, demonstrating that increasing levels of biotin can be detected using a concentration-based method. FIGS. 12a and 12b further illustrate that the resulting calibration can be used to quantify concentrations in unknown samples. Particularly, magnitude of impedance (Z) in the Bode plot (FIG. 12a) demonstrates a trend of increased impedance as higher concentrations of biotin were added to the bare (dark red) streptavidin-coated surface in 1:100 diluted urine samples in 50 mg NaCl solution. The increasing radius of the semicircle in the Nyquist plots (FIG. 12b) indicates an increase in the charge transfer resistance and more biotin binding.

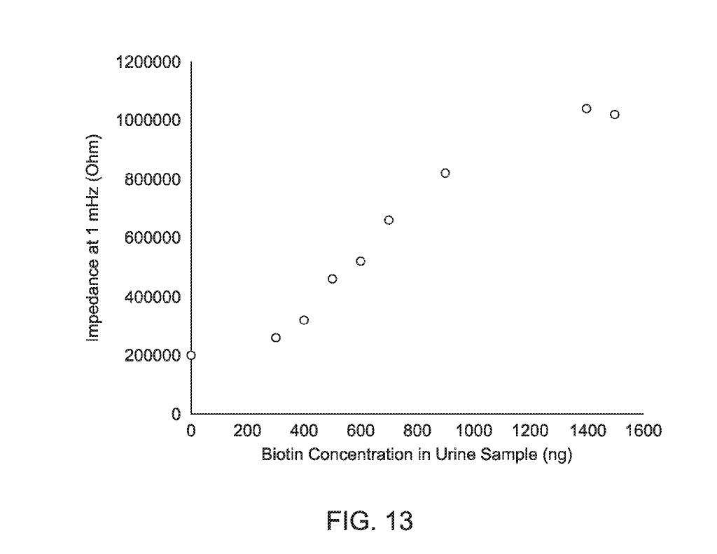

Plotting the impedance against the concentration of biotin in the diluted urine samples was linearly correlated within a physiologically relevant concentration range. Particularly, FIG. 13 shows impedance measurements at 1 mHz correlated to biotin concentration in spiked urine sample linearly, before saturating the streptavidin. The data indicates that correlation was strong (R²=0.99) within the range, with an average error of about 4.03% for the impedance at 1 mHz. The data shows that the impedance began to level off near 1.4 μg, likely indicating saturation of the streptavidin.

The correlations can be used to identify biotin correlations in unknown samples. In addition, using the same method for future biomarkers can help quantify vitamin, mineral, and other dietary nutrient levels in unknown samples. The quantifications can be normalized against creatinine to provide clinically relevant values for tracking and diagnosis.

### Example 2

### Creatinine Quantification in Diluted Urine Samples Using Cyclic Voltammetry

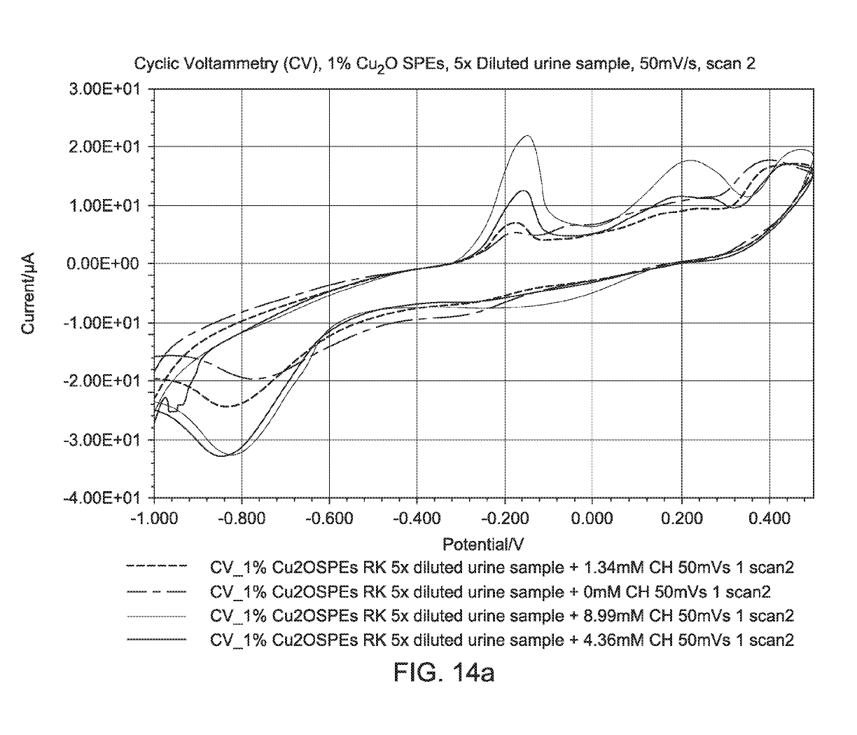

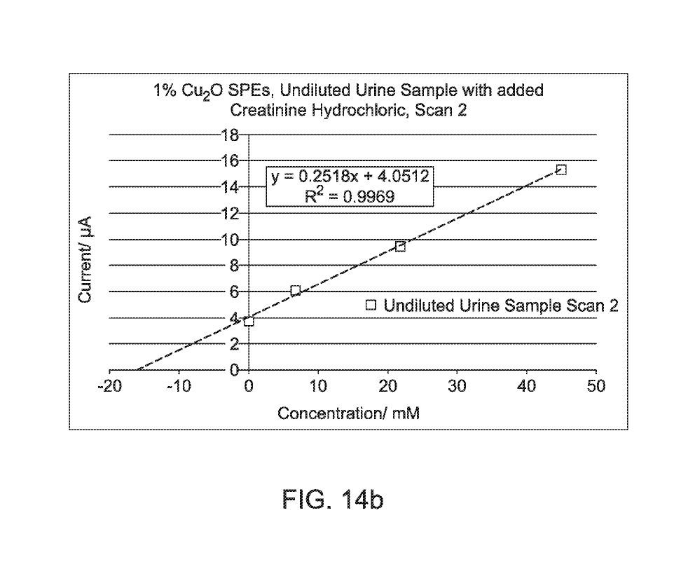

Voltammograms were taken before and after exposure of 1-5% copper I and copper II oxide embedded working electrodes to buffer and urine samples with and without creatinine. The data is shown in FIG. 14a and illustrates an increase in the peak at around −0.180 V upon increasing creatinine concentration. The linear correlation between creatinine concentration and peak height was shown to be strong (R²=0.99), as represented by FIG. 14b in urine samples.

### Example 3

### Urea Quantification in Diluted Urine Samples Using Cyclic Voltammetry

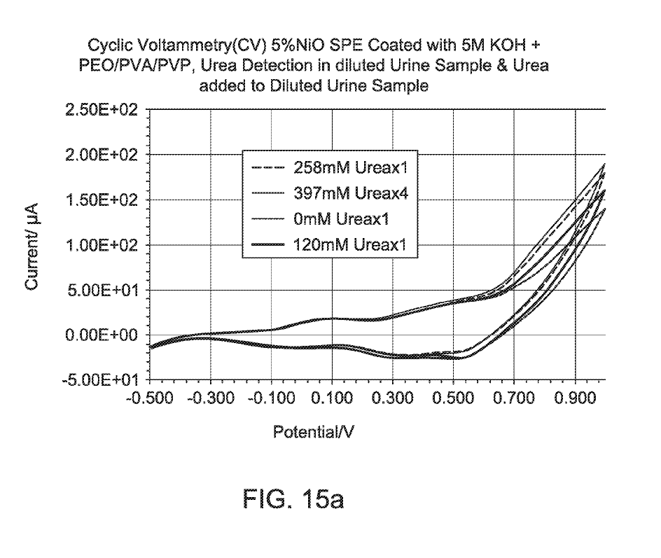

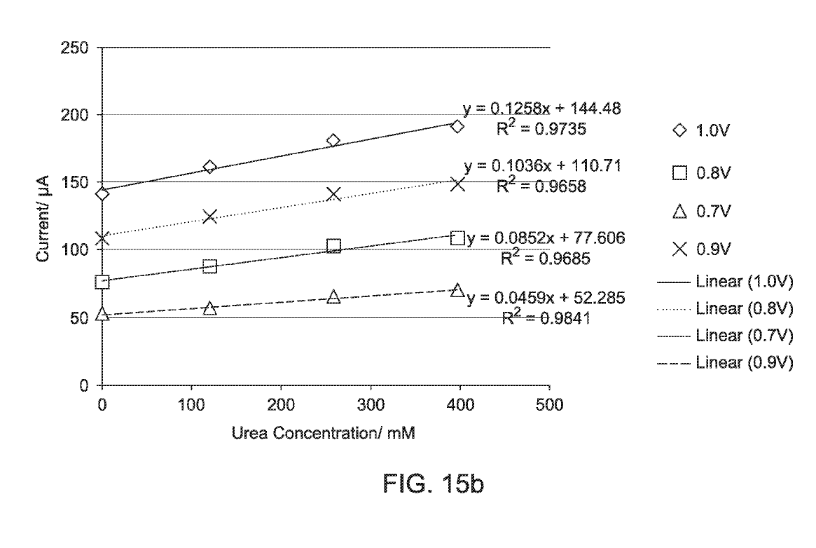

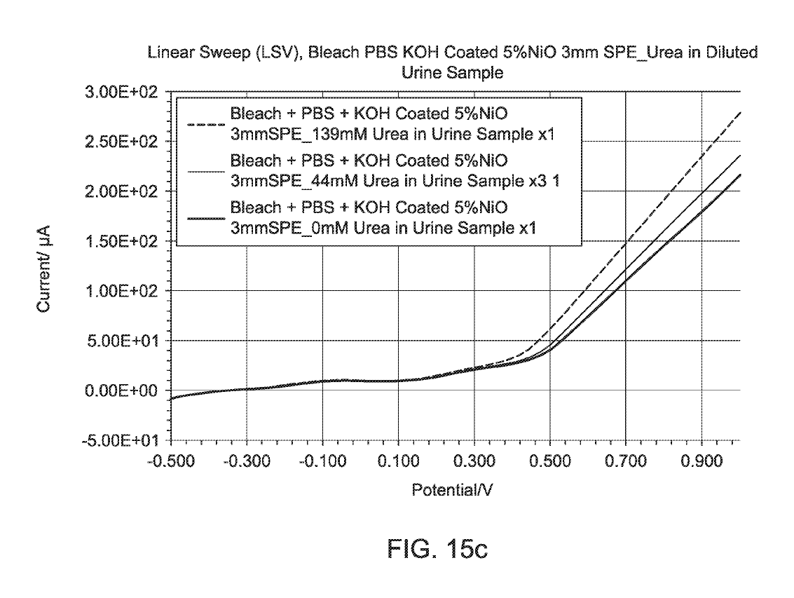

Voltammograms were taken before and after exposure of 1-5% nickel oxide embedded working electrodes to buffer and urine samples with and without urea. The data is shown in FIG. 15a and illustrates an increase in the scan wave beginning at about 0.3 V upon increasing urea concentration. The linear correlation between urea concentration and current at various voltages, as represented by FIG. 15b, was strong.

The reaction for urea detection is based on using a Ni electrode in alkaline medium for the electrolysis of urea:

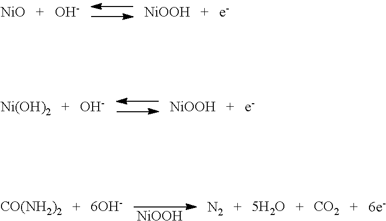

To coat 5% NiO modified multi electrodes strips, 3 ul of the mixture of 5M KOH, PVA, PVP and PEO were used and allowed to dry. A 50 ul drop of diluted urine sample (dilution by factor of 15 with deionised water) or creatinine and urea was added to diluted urine sample placed over coated multi SPEs. Linear voltammetric measurement was carried out from −0.5V to 1.5 using a scan rate of 25 mV/s, as shown in FIG. 15c. The data is given below in Table 1.

TABLE 1
Typical Data Analysis for Added Urea Detection in Diluted Urine

| | 0.60 V | 0.65 V | 0.70 V | 0.75 V | 0.80 V | 0.85 V | 0.90 V |
|---|---|---|---|---|---|---|---|
| | 392 mM | 392 mM | 392 mM | 392 mM | 392 mM | 392 mM | 392 mM |
| 1.00 | 36.99 | 48.46 | 64.24 | 81.87 | 100.04 | 80.13 | 135.07 |
| 2.00 | 43.83 | 58.88 | 78.52 | 99.90 | 121.62 | 96.88 | 163.50 |
| 3.00 | 37.86 | 50.31 | 67.72 | 86.76 | 105.71 | 85.69 | 143.84 |
| 4.00 | 41.64 | 54.47 | 72.78 | 93.27 | 114.27 | 92.38 | 155.07 |
| 5.00 | 41.64 | 55.84 | 75.59 | 96.48 | 117.37 | 96.69 | 160.13 |
| Avg. | 40.39 | 53.59 | 71.77 | 91.66 | 111.80 | 90.35 | 151.52 |
| Std. | 2.87 | 4.21 | 5.79 | 7.31 | 8.79 | 7.30 | 11.83 |
| Dev. | | | | | | | |
| CV | 7.10 | 7.85 | 8.07 | 7.97 | 7.86 | 8.08 | 7.81 |
| (%) | | | | | | | |
| | 0 mM | 0 mM | 0 mM | 0 mM | 0 mM | 0 mM | 0 mM |
| 1.00 | 34.17 | 41.83 | 53.15 | 67.46 | 82.20 | 59.14 | — |
| 2.00 | 36.79 | 45.17 | 57.43 | 72.91 | 88.97 | 64.81 | — |
| 3.00 | 38.81 | 47.57 | 60.19 | 77.27 | 95.29 | 67.44 | — |
| 4.00 | 35.04 | 43.11 | 55.01 | 69.63 | 84.84 | 61.75 | — |
| 5.00 | 38.22 | 46.74 | 59.06 | 75.77 | 93.66 | 66.44 | — |
| Avg. | 36.61 | 44.89 | 56.97 | 72.61 | 88.99 | 63.91 | — |
| Std. | 1.98 | 2.41 | 2.89 | 4.10 | 5.58 | 3.43 | — |
| Dev. | | | | | | | |
| CV | 5.42 | 5.37 | 5.07 | 5.65 | 6.27 | 5.37 | — |
| (%) | | | | | | | |
| Diff. | 3.78 | 8.71 | 14.80 | 19.05 | 22.81 | 26.44 | 27.83 |
| (392 | | | | | | | |
| mM - | | | | | | | |
| 0 | | | | | | | |
| mM) | | | | | | | |
| Sens. | 1.10 | 1.19 | 1.26 | 1.26 | 1.26 | 1.41 | 1.23 |

### Example 4

### Albumin Quantification in Diluted Urine Samples Using Cyclic Voltammetry

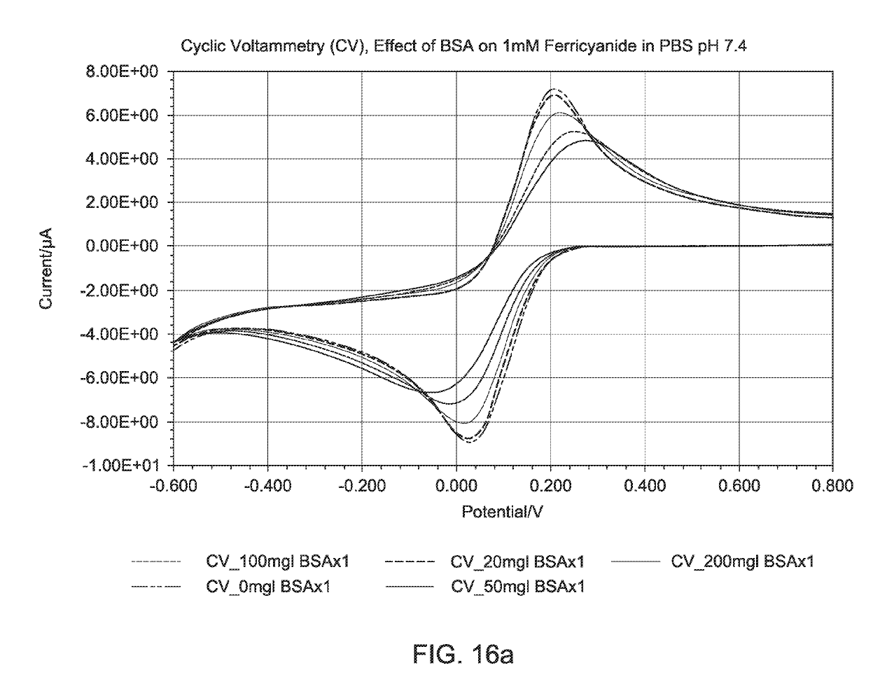

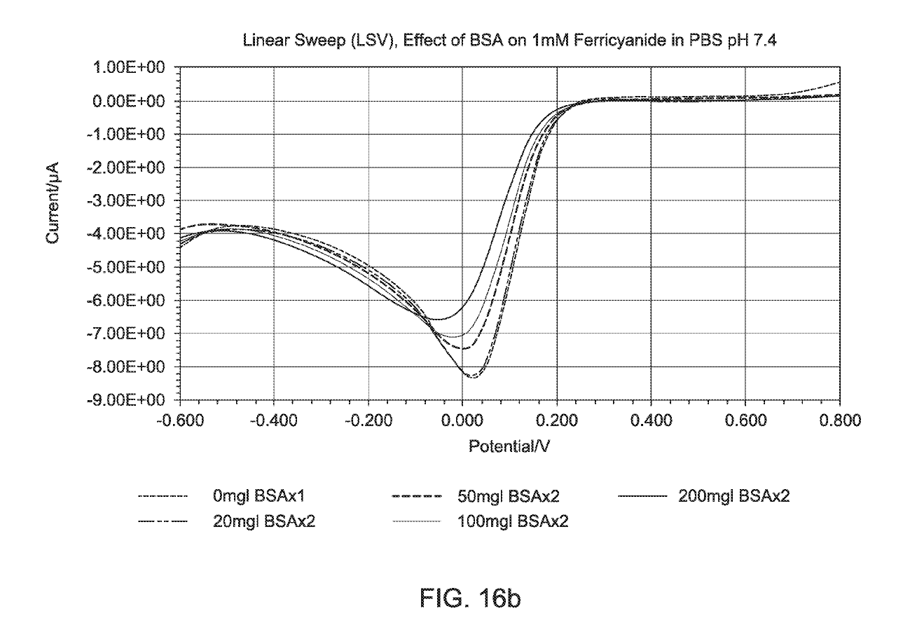

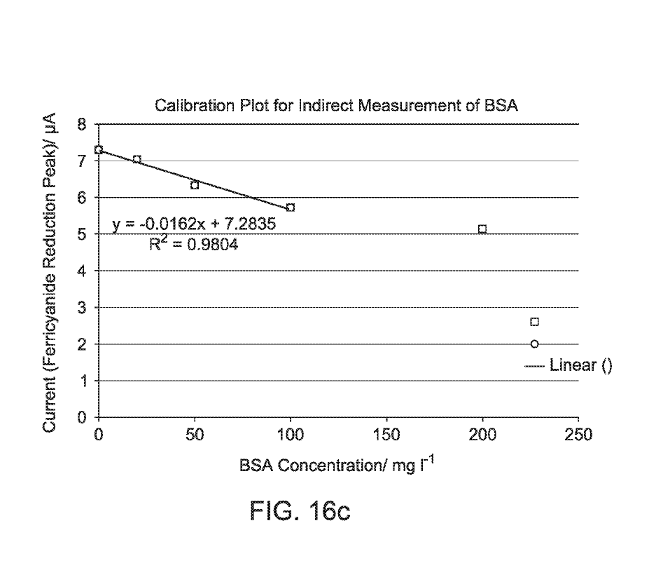

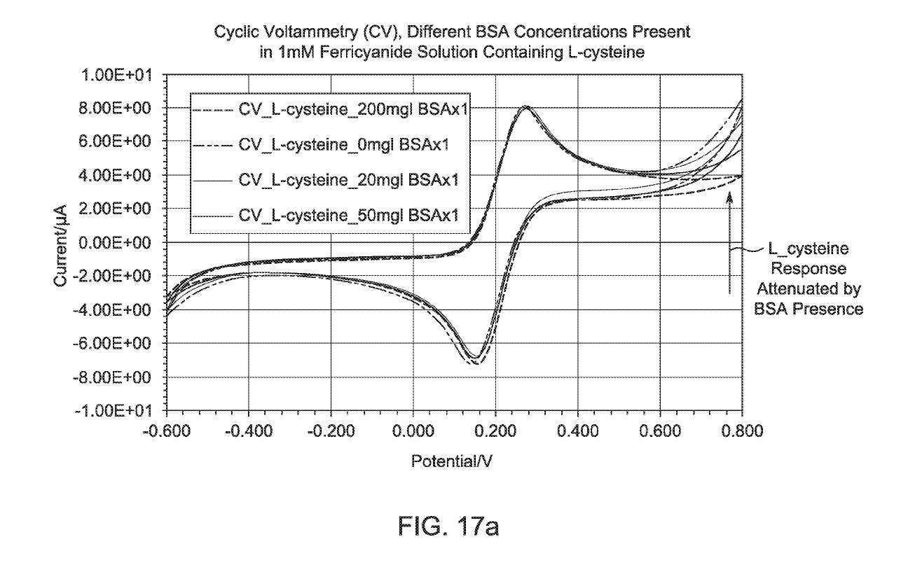

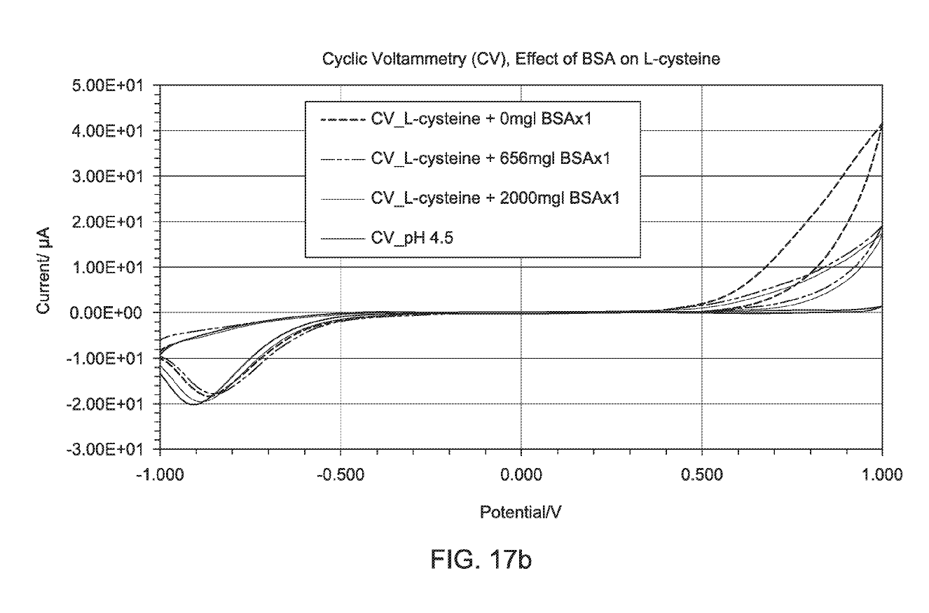

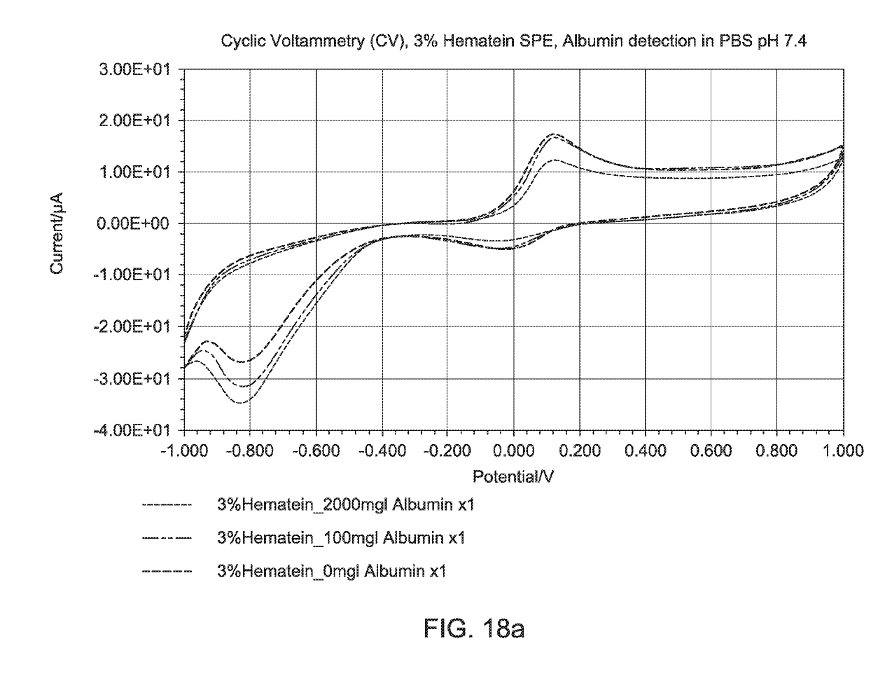

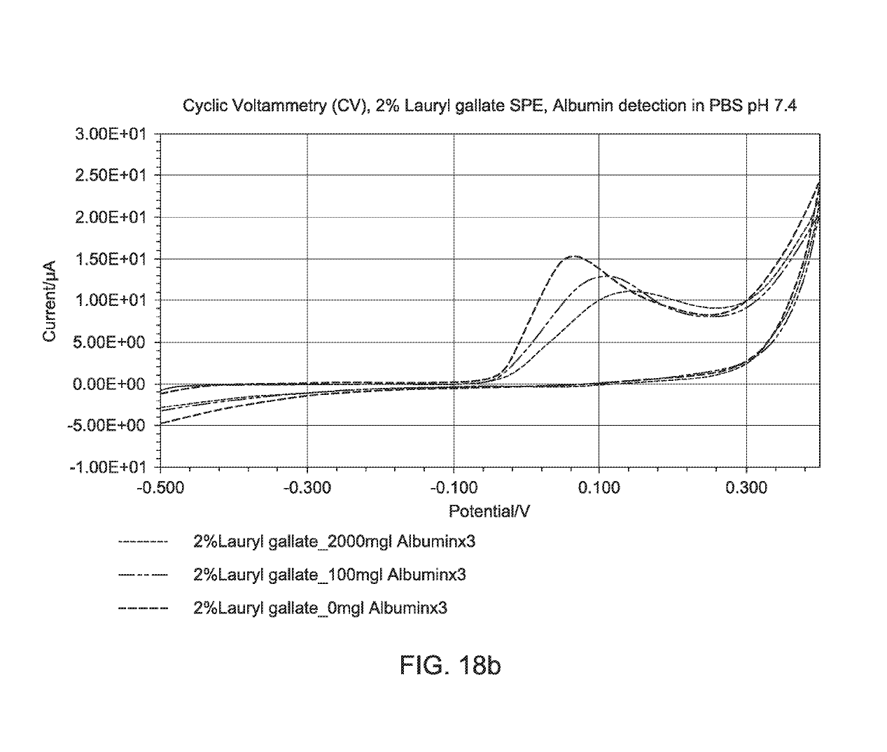

Voltammograms were taken before and after exposure of working electrodes with 1 mM ferricyanide deposited and dried on their surface to PBS buffer with and without albumin. The data is shown in FIG. 16a and illustrates an attenuation of the redox peak at around 0.2 V and 0.04 V upon increasing albumin concentration. A linear sweep voltammogram in FIG. 16b represents a similar progression in peak height upon increasing albumin concentration. The linear correlation between albumin concentration and current at various concentrations is represented by FIG. 16c. A working electrode modified with L-cystein showed a similar signal attenuation of the current wave from around 0.4 V to 0.8 V on working electrodes modified with (FIG. 17a) or without (FIG. 17b) 1 mM ferricyanide. A working electrode modified with 3% Hematein showed an increase in peak height at around 0.1 V upon increasing albumin concentration (FIG. 18a), and a working electrode modified with 2% lauryl gallate showed an increase in peak height and shift in peak voltage around 0-0.1 V upon increasing albumin concentration (FIG. 18b).

### Example 5

### Fructose Quantification in Diluted Urine Samples Using Linear and Cyclic Voltammetry

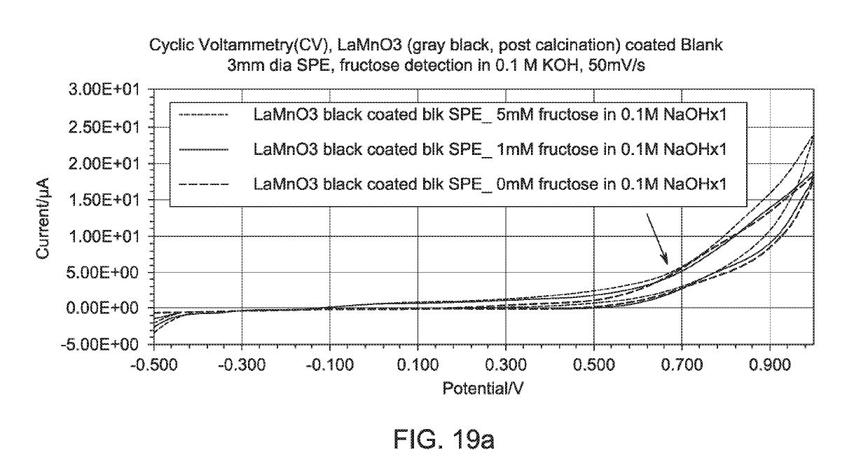

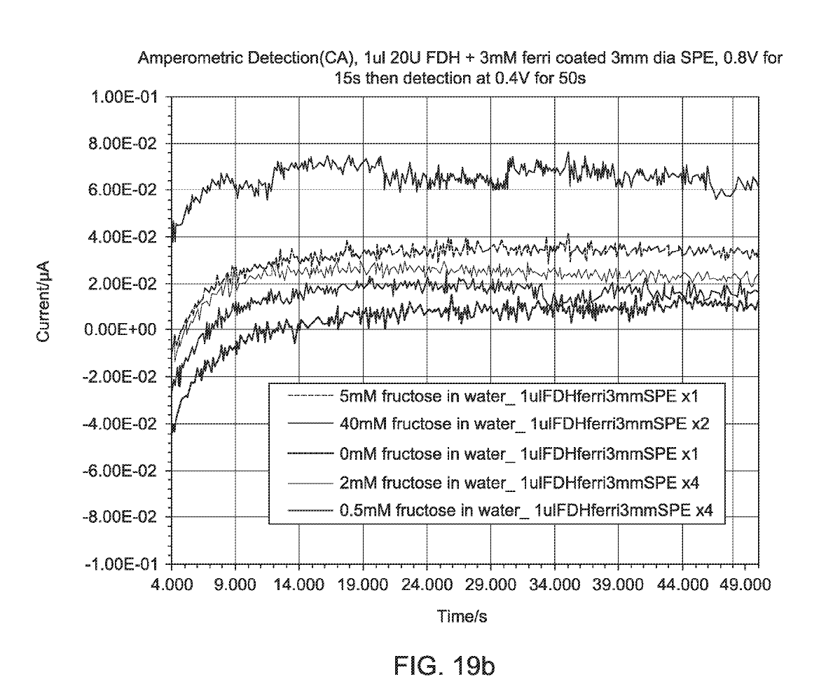

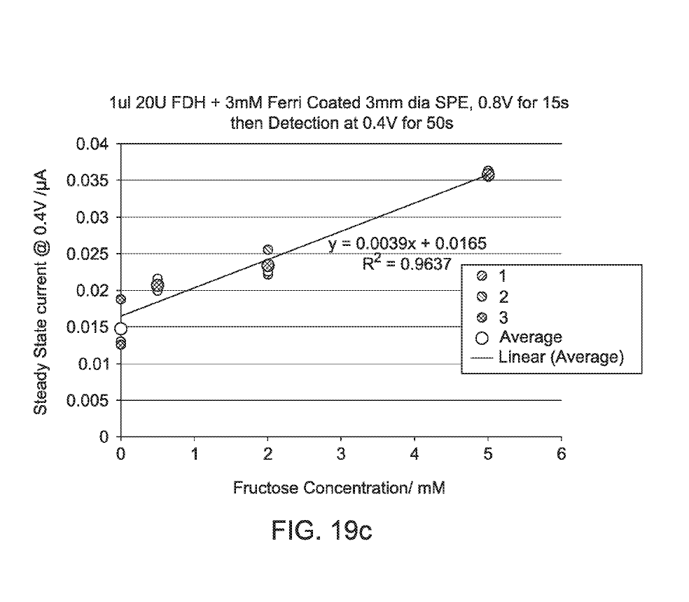

Voltammograms were taken before and after exposure of LaMnO₃ nanofiber coated working electrodes to buffer and urine samples with and without fructose. Other methods include incorporating LaMnO3 nanofibers into the carbon ink electrode material. The data, shown in FIG. 19a, shows an increase in the scan wave starting at around 0.1 V upon increasing fructose concentration.

LaMnO₃ fibers were synthesised by two steps: electrospinning and calcination process. To obtain a viscous gel, 1.08 g La(NO₃)₃.6H₂O, 0.61 g Mn(Ac)₂.4H₂O and 1.04 g PVP (MW 360,000) were slowly added into 10 mL DMF under vigorous magnetic stirring for 24 h. Then, the as-prepared gel was put in a plastic syringe with a stainless steel pinpoint, connecting to a high-voltage power supply for electrospinning. This process was performed with a high voltage of 15 kV and a distance of 10 cm between the spinneret and the collector. The flow rate of the gel was controlled at a rate of 0.5 mL h⁻¹ by using an injection pump. After electrospinning, the desiccation and stabilization of La(NO₃)₃/Mn(Ac)₂/PVP composite fibers was completed in a drying oven at 70° C. for 12 h. Finally, the as-prepared fibers were calcined in air at 600° C. with a heating rate of 2° C. min⁻¹ by using a furnace to get the LaMnO₃ sample.

Enzymatic methods were also tested, using 1 ul 20U fructose dehydrogenase+3 mM ferricyanide coated working electrodes. Chronoamperometry performed at 0.8V showed a favorable correlation to fructose concentration, as shown in FIGS. 19b and 19c.

### Example 6

### Vitamins B2, B9, and C Quantification in Diluted Urine Samples Using Linear and Cyclic Voltammetry

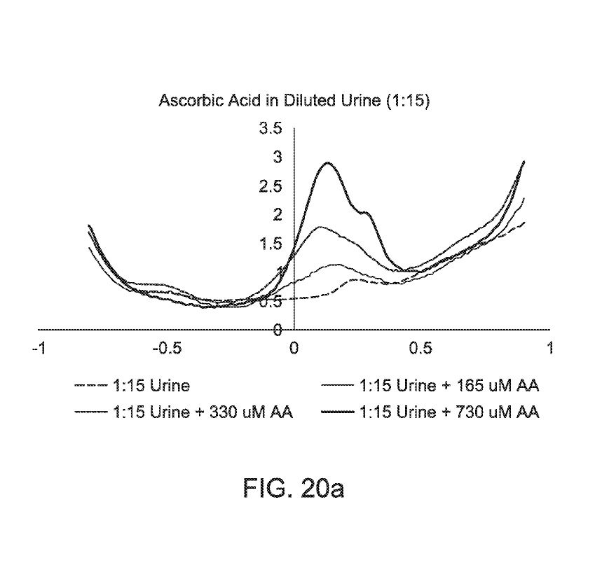

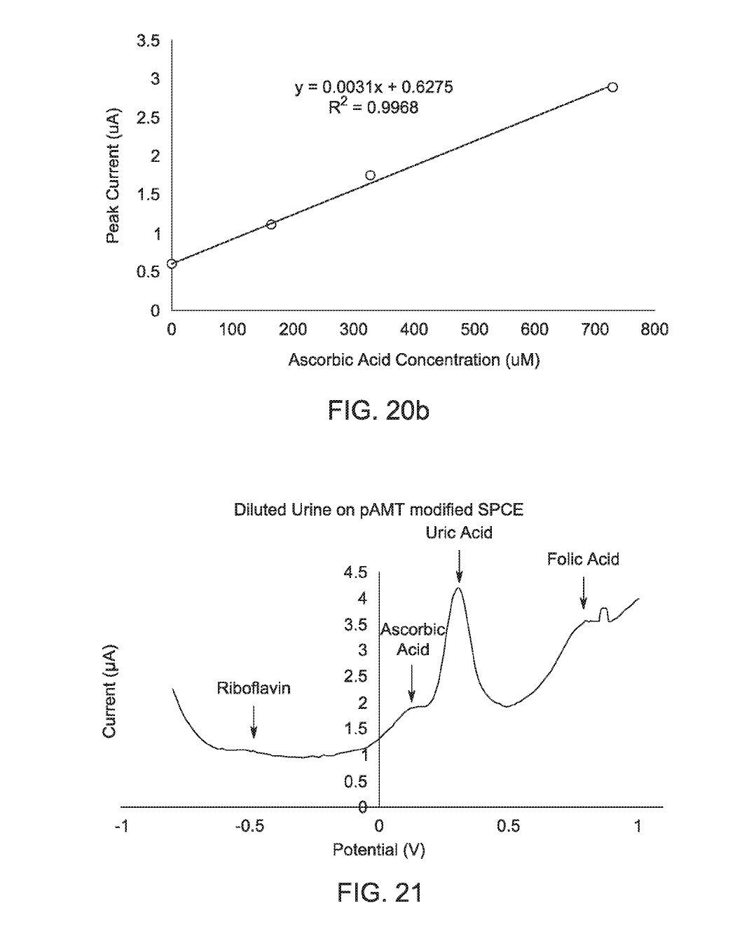

Voltammograms were taken before and after exposure of p-AMTa modified electrodes to buffer and diluted urine spiked with vitamins B2 (riboflavin), B9 (folic acid), and vitamin C (FIG. 21). Peaks for vitamin B2 were present around −0.5 V, vitamin C was present around 0.1-0.2 V, and vitamin B9 was present at around 0.75 V. As shown in FIG. 20a, increasing vitamin C (ascorbic acid) concentration results in increasing peak with a strong, linear correlation (FIG. 20b).

### Example 7

### Vitamins B2, B6, and C Quantification in Diluted Urine Samples Using Linear and Cyclic Voltammetry

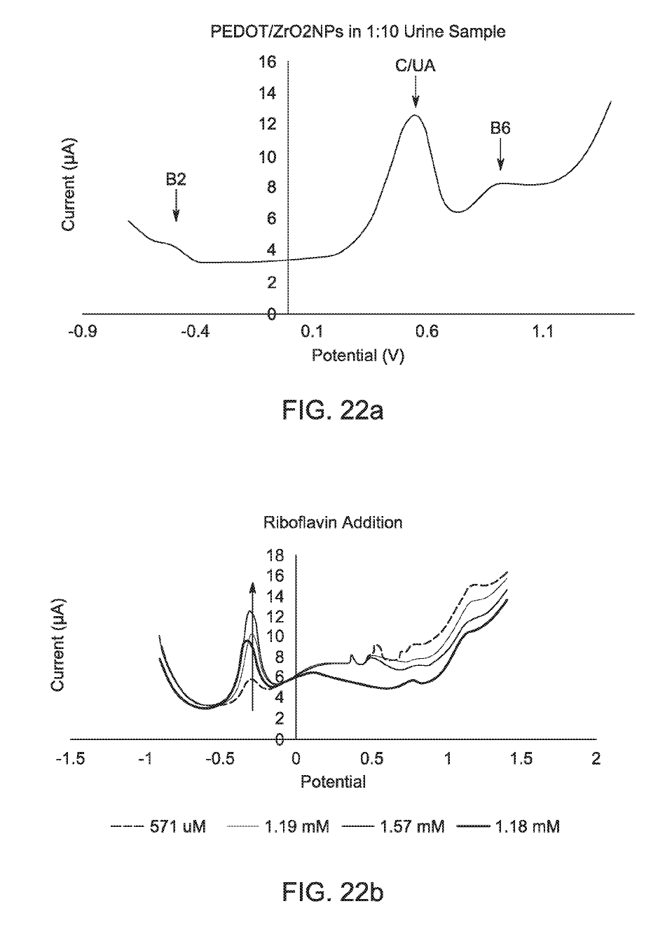

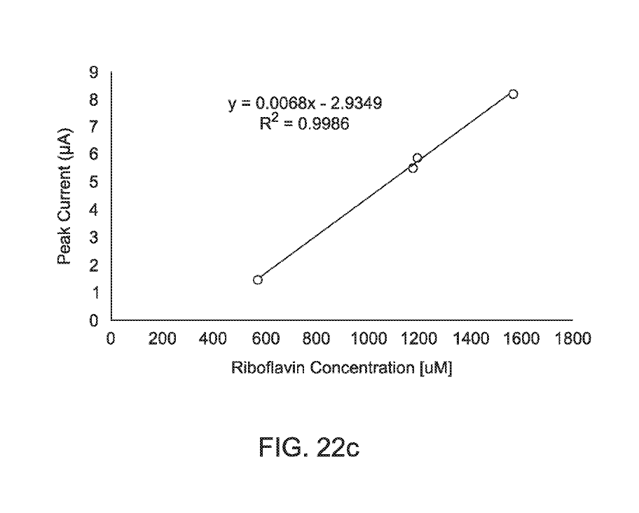

Voltammograms were taken before and after exposure of PEDOT/ZnNP modified electrodes to buffer and diluted urine spiked with vitamins B2 (riboflavin), B6, and vitamin C (FIG. 22a). Peaks for vitamin B2 were present around −0.5 V, vitamin C was present around 0.1-0.2 V, and vitamin B6 was present at around 0.8-0.9 V. As shown in FIG. 22b, increasing vitamin B2 (riboflavin) concentration results in increasing peak with a strong, linear correlation (FIG. 22c).

### CONCLUSIONS

Example 1 demonstrates the ability to detect vitamin B7 in urine samples for the purpose of quantifying an individual's micronutrient levels. Example 2 demonstrates the ability to detect creatinine in urine samples for the purpose of normalizing biomarker concentrations. Example 3 demonstrates the ability to detect urea in urine samples for the purpose of quantifying an individual's macronutrient levels. Example 4 demonstrates the ability to detect albumin in urine samples for the purpose of diagnosing chronic kidney disease and monitoring renal function. Example 5 demonstrates the ability to detect fructose in urine samples for the purpose of quantifying an individual's sugar intake levels. Example 6 demonstrates the ability to detect vitamins B2, C, and B9 in parallel in urine samples for the purpose of quantifying dietary micronutrient intake levels. Similarly, Example 7 demonstrates the ability to detect vitamins B2, C, and B6 in parallel in urine samples for the purpose of quantifying dietary micronutrient intake levels.

The method can be extended to include any and all vitamins, minerals, and other markers of dietary intake and/or nutritional health. In addition, the method can be extended to biomarkers of general health, disease, or other disorders. The data can be collected and tracked to better understand trends and improve the accuracy and reporting of dietary intake and nutritional health status. In some embodiments, the data can then be used to curate personalized nutritional solutions by creating personalized supplements with ingredients and ratios dictated by the patient's own urine sample biosensor measurements. It is envisioned that the data can be used to detect and assess clinically relevant health conditions, as well as population-based nutritional consumption patterns.

What is claimed is:

1. A method of obtaining data on the nutritional health of a subject, the method comprising:
contacting a solution comprising a subject's urine with a working electrode of a biosensor apparatus, the biosensor apparatus comprising:
at least one working electrode comprising an outside surface, wherein:
at least one affinity probe is present on the outside surface of the working electrode; or
the outside surface of the working electrode is free from bound affinity probe; and
an electrochemical spectroscopy analyzer;
performing electrochemical spectroscopy to generate data for the working electrode;
using the data to quantify an amount of at least one nutritional biomarker in the subject's urine; and
correlating the amount of the at least one nutritional biomarker to obtain nutritional health data.

2. The method of claim 1, wherein the affinity probe is an antibody, aptamer, affimer, hapten, enzyme, chemical, or combinations thereof.

3. The method of claim 1, wherein the affinity probe is bound to the working electrode using physioabsorption, covalent bonding, ionic bonding, physical entrapment, cross-linking, encapsulation, disulfide linking, chelation, metal binding, hydrophobic binding, or combinations thereof.

4. The method of claim 1, wherein the amount of the at least one nutritional biomarker comprises a correlation to intake of a dietary micronutrient, dietary macronutrient, dietary food group, total caloric intake, other dietary constituent, or combinations thereof.

5. The method of claim 1, further comprising comparing the subject's nutritional health data to recommended daily allowances, intake levels established by the subject, intake levels established by a dietitian, physician, or nutritionist, intake levels established algorithmically, or intake levels of segmented populations for a particular nutritional biomarker.

6. The method of claim 1, wherein the data is selected from impedance data, amperometric data, voltammetric data, potentiometric data, conductometric data, or combinations thereof.

7. The method of claim 1, wherein at least one electrode is used to specifically target creatinine.

8. A method of obtaining health data on the presence of a disease or disorder of a subject, the method comprising:
contacting a solution comprising a subject's urine with a working electrode of a biosensor apparatus, the biosensor apparatus comprising:
at least one working electrode comprising an outside surface, wherein:
at least one affinity probe is present on the outside surface of the working electrode;
or
the outside surface of the working electrode is free from bound affinity probe; and
an electrochemical spectroscopy analyzer;
performing electrochemical spectroscopy to generate data for the working electrode;
using the data to quantify the concentration of at least one biomarker associated with the presence of the disease or disorder in the subject's urine; and
correlating the concentration of the at least one biomarker to obtain health data related to the disease or disorder.

9. The method of claim 8, wherein the affinity probe is an antibody, aptamer, affimer, hapten, enzyme, chemical, or combinations thereof.

10. The method of claim 8, wherein the affinity probe is bound to the working electrode using physioabsorption, covalent bonding, ionic bonding, physical entrapment, cross-linking, encapsulation, di-sulfide linking, chelation or metal binding, hydrophobic binding, or combinations thereof.

11. The method of claim 8, wherein the health data:
Is used to monitor the disease or disorder; and
is used to diagnose the disease or disorder by correlating to known health data for the disease or disorder.

12. The method of claim 8, wherein at least one electrode is used to specifically target creatinine.

13. The method of claim 8, wherein the health data is selected from impedance data, amperometric data, voltammetric data, potentiometric data, conductometric data, or combinations thereof.

14. The method of claim 8, wherein the health data is collected by measuring:
impedance, using correlations between impedance and biomarker concentration to obtain biomarker concentration, and comparing the biomarker concentration to correlations between biomarker concentration and a disease or disorder;
voltammetric data, using current response to changing voltage;
amperometric data, using current response to a fixed voltage;
potentiometric data, using voltage response in relation to another electrode;
conductometric data, using electrical conductivity or current flow response due to a change ionic species separation; or
combinations thereof.

15. A method of diagnosing a condition or ailment in a subject, the method comprising:
contacting a solution comprising a subject's urine with a working electrode of a biosensor apparatus, the biosensor apparatus comprising:
at least one working electrode comprising an outside surface, wherein:
at least one affinity probe is present on the outside surface of the working electrode; or
the outside surface of the working electrode is free from bound affinity probe; and
an electrochemical spectroscopy analyzer;
performing electrochemical spectroscopy to generate data for the working electrode;
using the data to quantify the amount of at least one nutritional biomarker in the subject's urine;
correlating the amount of the nutritional biomarker to obtain nutritional health data; and
using the nutritional health data to diagnose a condition or ailment.

16. The method of claim 15, wherein the affinity probe is an antibody, aptamer, affimer, hapten, enzyme, chemical, or combinations thereof.

17. The method of claim 15, wherein the nutritional biomarker comprises a correlation to a clinical pathology and is used to diagnose a pathology, monitor a pathology or both.

18. The method of claim 15, wherein the affinity probe is bound to the working electrode using physioabsorption, covalent bonding, ionic bonding, physical entrapment, cross-linking, encapsulation, di-sulfide linking, chelation or metal binding, hydrophobic binding, or combinations thereof.

19. The method of claim 15, wherein the nutritional health data is collected by:
measuring impedance, using correlations between impedance and biomarker concentration to obtain biomarker concentration, and comparing the biomarker concentration to correlations between biomarker concentration and dietary intake;
voltammetric data, using current response to changing voltage; amperometric data,
using current response to a fixed voltage;
potentiometric data, using voltage response in relation to another electrode;
conductometric data, using electrical conductivity or current flow response due to a change in ionic species concentration; or
combinations thereof.

20. The method of claim 15, wherein the nutritional health data is used to:
provide customary food, dietary, or both food and dietary suggestions;
develop a custom medication; or
both.
